(19) United States
(12) Patent Application Publication
(10) Pub. No.: US 2020/0069239 A1
(43) Pub. Date: Mar. 5, 2020

(54) DEVICE, SYSTEM AND METHOD FOR MONITORING NEUROLOGICAL FUNCTIONAL STATUS

(71) Applicant: MUSC Foundation for Research Development, Charleston, SC (US)

(72) Inventors: Nancey Trevanian Tsai, Hanahan, SC (US); Mark E. Semler, Mt. Pleasant, SC (US)

(21) Appl. No.: 16/613,522

(22) PCT Filed: May 15, 2018
(86) PCT No.: PCT/US18/32666

§ 371 Date: Nov. 14, 2019

Related U.S. Application Data
(60) Provisional application No. 62/506,160, filed on May 15, 2017.

(51) Int. Cl. A61B 5/00; A61B 5/16; A61B 5/11; G16H 40/67; G16H 20/70; G16H 50/20

(52) U.S. Cl. CPC A61B 5/4076; A61B 5/162; A61B 5/163; A61B 5/1104; G16H 50/20; A61B 5/7475; A61B 5/0077; G16H 40/67; G16H 20/70; A61B 5/743

(57) ABSTRACT

A device for measuring eyelid movement in a human subject comprises a housing, at least one stimulator mounted to the housing, and a camera. The at least one stimulator is configured to provide stimulus to one or both eyes of the subject. The camera is configured to collect information related to movement of one or both eyes of the subject. The device also includes a user interface that is configured to control the at least one stimulator and display information collected by the camera.

### CROSS-REFERENCE TO RELATED APPLICATIONS

This application claims priority to U.S. provisional application No. 62/506,160 filed on May 15, 2017 incorporated herein by reference in its entirety.

### BACKGROUND

Mild traumatic brain injury (mTBI), sometimes referred to as a concussion, mild brain injury, mild head injury (MHI), or minor head trauma, is the most common type of traumatic brain injury. The rate at which mTBI occurs is not accurately known, which may be due to the subjective nature of its detection and diagnosis, and the possibility that occurrences of mTBI are being under-reported. Some estimates suggest that mTBI occurs in more than six (6) per one thousand (1,000) people per year. Common causes of concussions are sports injuries, bicycle accidents, car accidents and falls. Concussions caused by sports injuries and bicycle injuries most commonly occur in children and young adults, and those caused by car accidents and falls most commonly occur in adults and the elderly.

The prevailing definition of a concussive injury is disruption of normal brain activity caused by rapid acceleration and deceleration of brain matter. Concussion can occur with or without the loss of consciousness and its impact on an individual's health is wide-spread, including physiological and metabolic changes within the brain affecting cognitive and emotional function. Athletes participating in collision/contact sports are at heightened risk for suffering a concussive event, in fact, roughly 250,000 individuals age 19 and younger visit a US emergency department for a sport or recreation-related concussion annually. Of concern is the potential for repeated concussive events, as demonstrated in a study of collegiate football players that found of the 6.3% of concussed players, 14.7% went on to experience a second concussion. Repeated head impacts can result in second impact syndrome or chronic traumatic encephalopathy both with the potential for long-term disabilities or death. Thus, the ability to accurately diagnose concussions and identify athletes at risk for long-term complications is an important clinical goal.

Despite the number of athletes affected, the ability for sports medicine professionals to confidently diagnosis and monitor concussion recovery is a challenge recognized by organizations such as the National Athletic Trainers Association (NATA). Current methods of diagnosing concussion typically include self-report and a battery of tests, including neurocognitive function and balance performance, aimed at evaluating symptoms associated with concussions. Of these, only one adult and one pediatric test evaluating neurocognitive function are FDA approved for concussion diagnosis. However, research on neurocognitive testing shows poor validity across age groups and low test-retest reliability, with 22-46% of healthy controls being misclassified as impaired. This issue is compounded by the admission of student and professional level athletes that they have hidden or would hide the symptoms of a concussion to avoid missing participation time. As such, there remains a clinical need for an objective diagnostic test that cannot be cheated.

Part of the problem of the diagnosis of mTBI is that there are little differences between the diagnostic criteria and the manifest symptoms. mTBI implies decreased cognitive function and denotes change in personality and behaviors that are uncharacteristic of the person who has sustained a mTBI. While there are known systems and methods for identifying or measuring cognitive function in a subject, there are currently no devices and methods that objectively measure, on a near real-time basis in the field (e.g., the playing field, battlefield, site of an automobile accident, etc.), the likelihood of altered brain reflexes and/or physiology associated with a neurological condition within a subject.

### SUMMARY

In one embodiment, an apparatus is provided for detecting parameters associated with an eye by stimulus thereto. The apparatus includes at least one stimulator, at least one sensor, and a user interface. The at least one stimulator provides stimulus to one or both eyes of a subject. The at least one sensor is configured to detect a parameter of one or both eyes. The user interface is configured to control the at least one stimulator and display information detected by the at least one sensor.

In another embodiment, an apparatus for mounting to a stimulus device used for detecting parameters associated with an eye by stimulus thereto. The apparatus comprises a unit having a channel that extends therethrough from a first opening to a second opening. The first opening extends in a first direction, and the second opening extends in a second direction. The first direction is angularly offset from the second direction.

In yet another embodiment, a method for detecting parameters associated with an eye by stimulus thereto using device that creates the aforementioned stimulus device. The method comprises stimulating one or both eyes of the subject so as to cause an involuntarily response in the subject, measuring a time period from the stimulating step to when one or both eyes initiates a parameter associated with the eye, and displaying information that identifies the time period. Alternatively, or in addition, parameters associated with eyelid movement, pupillary response of the subject, and/or reflective light patterns in response to a light stimulus may be measured.

### BRIEF DESCRIPTION OF THE DRAWINGS

FIGS. 1A-1D are diagrams of an example embodiment of a blink reflex device;

FIG. 2A is a perspective view of an embodiment of the blink reflex device;

FIG. 2B is a perspective view of a cross section of the blink reflex device taken along line B-B of FIG. 2A;

FIG. 2C is a top view of the blink reflex device;

FIG. 2D is a cross section of the blink reflex device taken along line D-D of FIG. 2C;

FIG. 2E is a cross section of the blink reflex device taken along line E-E of FIG. 2C;

FIG. 3 is a perspective view of an alternate embodiment of the blink reflex device showing a subject making use thereof;

FIG. 4 is a diagram of an example environment in which the blink reflex device shown in FIGS. 1A-1D may be implemented;

FIG. 5 is a diagram of example components of a blink reflex device of FIG. 1A-1D;

FIG. 6A is a diagram of an example eyelid tracking scheme associated with measuring a blink reflex of a subject;

FIG. 6B is a diagram of example stages of a blink of an eye of a subject from which a blink reflex can be measured;

FIG. 6C is a diagram of an example blink reflex response associated with a subject;

FIG. 7 is an illustration of an example blink reflex device;

FIGS. 8A-8D are diagrams of different types of example blink reflex responses associated with a subject;

FIG. 9 is a diagram of an example blink reflex response, associated with a subject, that includes data to be removed and/or filtered from the example blink reflex response;

FIG. 10 is a flowchart of an example process for determining whether a subject suffers from brain injury or a degenerative neurological condition;

FIG. 11 is a diagram of an example data structure that may store information associated with a blink reflex of a subject; and

FIG. 12 is a diagram of an example data structure that stores information associated with a change in blink reflex of a subject.

FIG. 13 is an image of the experimental system housing unit and software interface according to one embodiment. Tubing connected to the left end of the housing unit delivers a puff of compressed air to the subject's eyes.

FIGS. 14A-14C illustrate time-displacement profiles of upper lid movement during and after a stimulated blink. FIG. 14A illustrates a baseline blink reflex time-displacement profile. FIG. 14B illustrates a blink reflex time-displacement profile after active play. Individual latency is increased. Differential latency is decreased. Log of number of oscillations is less. FIG. 14C illustrates a blink reflex time-displacement profile after a head impact causing a concussive event. Individual latency is decreased. Differential latency is increased. Log of number of oscillations is increased.

FIG. 15 is a spaghetti plot of measured reflex parameter changes due to active play or head impact.

FIG. 16 is a table of estimated mean differences in reflex parameters in active play and head impacted athletes, along with corresponding standard error values.

### DETAILED DESCRIPTION

The devices and methods described herein may be used to determine whether a human subject suffers from impaired neurological function based on a change in a blink reflex, blink period, or other brain reflex of the subject. Impaired neurological function may result from a traumatic event, a head impact, a brain injury, such as mTBI, Second Impact Syndrome (SIS), a degenerative neurological condition such as Alzheimer's disease and Parkinson's disease (hereinafter collectively referred to as a “neurological condition”), or may be due to other causes (e.g., due to fatigue, exhaustion, a developmental abnormality, narcotics, alcohol, or an illness other than a neurological illness, etc.). In the event that it is determined that the subject may suffer from a neurological condition, the devices and methods may enable detection of the level of severity of such a neurological condition.

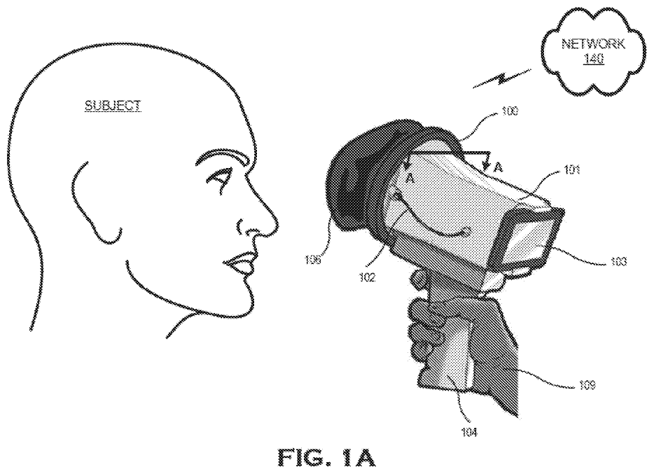

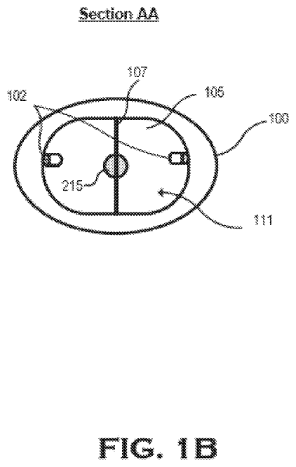

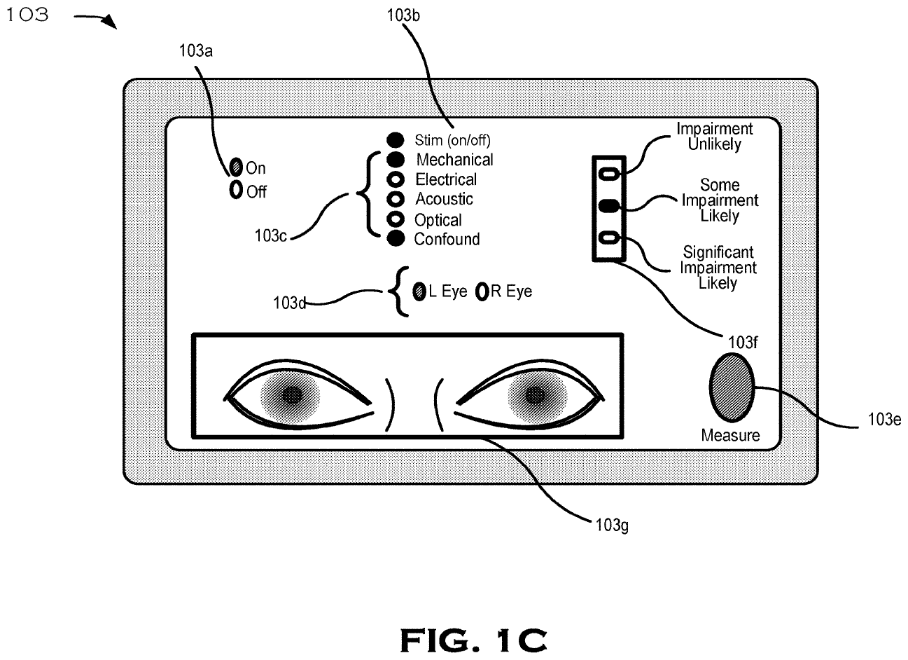

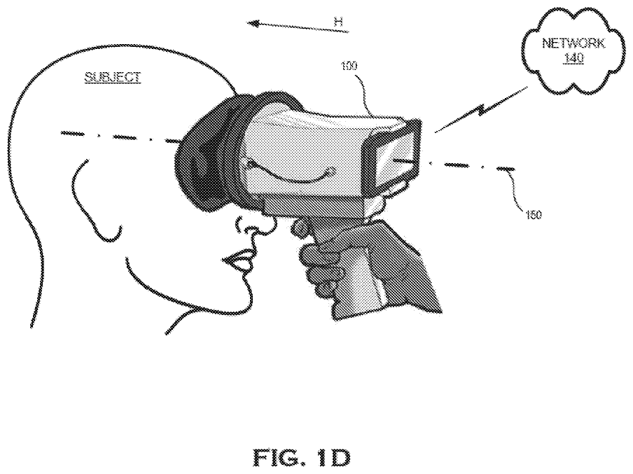

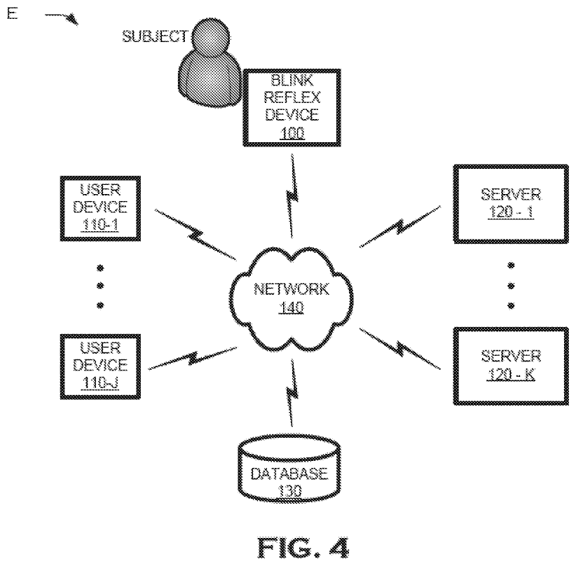

FIGS. 1A-1D are diagrams of an example blink reflex device 100. As shown in FIG. 1A, blink reflex device 100 may include a housing 101, a stimulator 102, and a sensor 215 (shown in FIG. 1B), such as a camera. With reference to FIG. 4, blink reflex device 100 may communicate with server 120 and/or database 130 via network 140. Blink reflex device 100 may include a collection of components such as, for example, a user interface 103, a handle 104, and a screen 105 (shown in FIG. 1B).

Device 100 may include a flexible material 106 attached to the housing 101 configured to fit against the face, head, or neck of the subject. Flexible material 106, together with housing 101, defines a cavity 111 within which the stimulators 102, sensor 215, and screen 105 are disposed. Flexible material 106 conforms to the shape and contours of the subject so as to create a temporary seal between the subject and blink reflex device 100. The seal may enable stimulator 102 and or sensor 215 to operate with minimal external stimulation or light. Screen 105 may also, or alternatively, minimize the likelihood that the subject is distracted by objects or activities that are outside the cavity 111. Handle 104 may include a rigid material that is part of or connected to housing 101, and configured to be held by an operator 109 of the blink reflex device 100. User interface 103 allows the operator 109 to operate and/or control blink reflex device 100.

By way of example, the operator 109, of blink reflex device 100, may place blink reflex device 100 against the subject's face to detect and monitor one or both eyes of the subject to measure and/or obtain information associated with a blink reflex and/or blink period of the subject (e.g., as shown in FIG. 1D). FIG. 1B, which depicts section AA of blink reflex device 100 as shown in FIG. 1A, depicts a pair of stimulators 102, sensor 215, screen 105, and a divider 107. Stimulators 102 may provide mechanical stimuli (e.g., a puff of fluid, etc.) and/or some other type of stimuli (e.g., light, acoustic, electrical, etc.) to the subject. Sensor 215 measures the blink reflex and/or blink period of the subject. Sensor 215 may also, or alternatively, detect parameters associated with the eye based on a stimulus applied thereto, including, for example, eye movement, eyelid movement, and/or pupillary response of the subject.

As set forth generally above, stimulator 102 may include one or more components to provide mechanical, electrical, optical, and/or acoustic stimulation to a subject, to trigger a blink reflex in the subject. The stimulation may excite certain neural pathways in the brain and/or nervous system of the subject, which may trigger the blink reflex. For example, optical stimulation (e.g., due to a beam or flash of light directed at the eye of the subject) may stimulate the superior colliculus structure and/or some other structure in the brain to cause the subject to involuntarily blink. Additionally, or alternatively, mechanical stimulation (e.g., a puff of air to the eye, a pin prick in close proximity to the eye, etc.) and/or electrical stimulation may excite the corneal reflex and/or some neurological structure of the subject causing the subject to involuntarily blink. Additionally, or alternatively, acoustic stimulation (e.g., a sudden loud tone, noise, music, etc.) may stimulate the inferior colliculus structure and/or some other structure in the brain to cause the subject to involuntarily blink or elicit some other involuntary brain reflex. Stimulator 102 may output the stimulation based on an instruction received from processing unit 400 (shown in FIG. 4) and/or an operator of blink reflex device 100. Stimulator 102 may also, or alternatively, include a device to confound, by distraction, sensitization or other means, the subject to attenuate a tendency by the subject to anticipate or habituate to certain stimuli, which may affect the integrity of the blink reflex data and/or other brain reflex data.

Divider 107 forms a barrier between a right side and left side of the cavity 111 defined by the flexible material to preclude a stimulus, provided by one of the stimulators 102, from inadvertently stimulating the eye that is closest to the other stimulator 102. Divider 107 is configured such that sensor 215 can measure the blink reflex, blink period, eye movement, or pupillary response of one or both eyes of the subject. Divider 107 may be made of a flexible material that conforms to the shape of the subject's face, nose, forehead, etc. Divider 107 may also, or alternatively, be removable.

With reference to FIG. 1B, screen 105 may be used to display instructions for the subject during a confounding operation or stimulation, or may be used to display a target at which the subject is to stare during a measurement, etc. Screen 105 may enable questions, lights, etc. associated with a confounding operation to be displayed for the subject. Screen 105 may provide means of optical stimulus in place of or in combination with stimulus provided by stimulator 102.

As shown in FIG. 1C, user interface 103 may include a collection of buttons, fields and/or indicators, such as a power button 103a, a stimulator button 103b, a stimuli selector button 103c, an eye selector button 103d, a measure button 103e, an indicator 103f and a subject field 103g. User interface 103 may receive information from processing unit 400 (shown in FIG. 4) and may display the received information. User interface 103 may receive information from an operator 109 of blink reflex device 100 and may provide the entered information to processing unit 400. The number of components, buttons, fields and/or indicators, illustrated in FIG. 1C is provided for explanatory purposes only. In practice, there may be additional components, fields, buttons, and/or indicators; fewer components, fields, buttons, and/or indicators; different components, fields, buttons, and/or indicators; or differently arranged components, fields, buttons, and/or indicators than illustrated in FIG. 1C.

Power button 103a may include one or more buttons that enable the blink reflex device 100 to power up or power down. Stimulator button 103b enables the operator 109 to control blink reflex device 100 to provide stimulus to the subject or to preclude stimulus from being provided to the subject.

Stimuli selector button 103c enables selection of a type of stimulus (e.g., mechanical, electrical, acoustic, optical, etc.) to be provided to the subject by blink reflex device 100. Stimuli selector button 103c may also, or alternatively, enable control of whether or not blink reflex device 100 will provide a confounding operation to the subject. Eye selector button 103d may enable selection of the left eye, right eye, or both eyes from which information associated with a blink reflex and/or blink period is to be obtained by blink reflex device 100. Measure button 103e, when selected by the operator, causes blink reflex device 100 to measure the blink reflex and/or blink period of the subject in a manner that includes the type of stimuli with or without confounding as selected by the operator using stimuli selector button 103c. Indicator 103f may include one or more lights, light emitting diodes, a display, a user interface, speaker, etc. that enables blink reflex device 100 to output an indication, notification, and/or sound that can be viewed or heard by the operator 109 of blink reflex device 100 that identifies whether the subject suffers from a neurological condition and/or a level of severity of such a neurological condition. For example, if blink reflex device 100 determines that the subject likely suffers from some brain injury or degenerative neurological condition that is not significant, blink reflex device 100 may cause a light, indication, notification, etc. to be lighted or displayed in a manner that indicates that the subject suffers from some brain injury or degenerative neurological condition. Subject field 103g may include an image or video of the subject as seen by sensor 215 before, during, and/or after measurements are taken on the subject.

FIG. 1D is an illustration of the blink reflex device 100 being used to take a blink reflex and/or blink period measurement from the subject. As shown in FIG. 1D, the operator 109 may place blink reflex device 100 against the face of the subject to obtain information associated with the blink reflex and/or blink period in a manner described above. The subject may be spaced from the blink reflex device 100 in a horizontal direction H. The horizontal direction H is parallel to a central axis 150 that extends from the user interface 103 to the subject.

Blink reflex device 100 may, for example, be configured to measure a response associated with an eye blink of a subject (hereinafter the “blink reflex”). The blink reflex (described in greater detail herein) generally corresponds to measurements of time, position and rates of eyelid movements.

Blink reflex device 100 may be configured to measure a period of time that it takes for the subject to blink his or her eye (hereinafter, the “blink period”). The blink period may be measured on the subject's stimulated blink; an intentional and voluntary blink; and/or an involuntary, unintentional or subconscious blink. The blink period may be measured from when the subject starts to blink (e.g., when the eyelid, in an open state, begins to close) to when the subject stops the blink and the eye of the subject returns to the open state (e.g., when the eyelid, returning from a closed state, stops opening). Blink reflex device 100 may measure a time period from when stimulation is received within the proximity of the eye of the subject to when the subject initiates or begins to blink (e.g., when one or more of the subject's eyelids, in an open state, begin to close) in response to the stimulation (hereinafter “individual latency”). Blink reflex device 100 may be configured to measure a time discrepancy between movements of the subject's two eyelids (“differential latency”). The time discrepancy may be measured as the time difference between stimulation and when each eyelid starts moving. Blink reflex device 100 may also be configured to determine the number of times that the subject's eyelids oscillate during a blink period (“oscillations”). An oscillation is a cycle of down and up movement of one or both eyelids after a stimulated blink. One or more oscillations may occur in response to stimulation. Blink reflex device 100 may also be configured to detect changes in the open lid position of one or both of the subject's eyelids (“tonic lid position”).

Blink reflex device 100 may also, or alternatively, be configured to detect when the subject exhibits an abnormal blink and may reject, discard, and/or ignore any data associated with a blink reflex measurement of the abnormal blink or other non-reflex closure or movement of the eye. An abnormal blink may occur when the eye of the subject does not fully return to the open state, does not fully close, remains closed for a prolonged time period (e.g., greater than 2 times, 5 times, 10 times, 15 times, etc. of a normal blink period) (sometimes referred to as a “micro-sleep”).

Blink reflex device 100 may be configured to measure the blink reflex for either eye (unilateral) or both eyes (bilateral) of the subject based on an intentional blink by the subject (e.g., a conscious blink in response to a command), a spontaneous blink of the subject (e.g., an unconscious blink to moisten or lubricate the eye), or a reflexive blink of the subject in response to one or more different types of stimulation (e.g., electrical, mechanical, acoustic, optical, or some other type of stimulation) directly to the eye, eye lid, eye lashes, or proximity of the eye (e.g., within ¼, ½, 1, 2, etc. inches of the eye or eyelid). The different types of stimulation may trigger different neural pathways within, and/or neurological functions of, the brain to cause the blink reflex. Thus, measuring the blink reflex using different types of stimulation may enable a type of neurological impairment within the brain to be identified and/or a specific location or structure, within the brain, that has been injured or impaired, to be identified.

Blink reflex device 100 may be configured to compare the measured blink reflex, blink period, or a brain reflex to a baseline blink reflex, blink period, or some other brain reflex to identify an amount difference between the measured blink reflex, blink period, or brain reflex and the baseline blink reflex, blink period, or some other brain reflex, respectively. The baseline measurement may correspond to a blink reflex, blink period, or brain reflex that is measured from the subject at a time when the subject is known not to be suffering from a neurological condition. For example, the baseline blink reflex, blink period, or brain reflex may be measured prior to the occurrence of a traumatic event, such as a blow to the head of the subject (e.g., on the field of play, on the battlefield, in a car accident, a physical altercation, etc.). Alternatively, the various baseline measurements described herein may be obtained by other means, including but not limited to a population average, an average based on a subset of the population similar to the subject, an average based on a regional population, information obtained from medical journals or treatises, or a measurement taken at a time when the subject is known not to be suffering from a neurological condition. In some embodiments, multiple sources of baseline measurements may be combined to further refine one or more baseline measurements. Device 100 may also, or alternatively, be configured to determine whether the subject suffers from a neurological condition and/or the severity thereof based an amount of change between the measured blink reflex, blink period or brain reflex, and the baseline blink reflex, blink period and/or some other brain reflex, respectively. Additionally, or alternatively, the blink reflex device 100 may enable the type of neurological condition and/or specific locations in the brain that have be injured to be identified based on a respective amount of change of the blink reflex, blink period and/or brain reflex for each of the different types of stimulation. Device 100 may also, or alternatively, enable the type of neurological condition and/or specific locations or structures of the brain that have been injured to be identified based on differences in the blink reflex and/or blink period between the left and right eye. Over time, device 100 may be configured to track changes in the baseline blink reflex, blink period, and/or brain reflex as a subject ages or is repeatedly exposed to brain or neurological trauma.

Additionally, or alternatively, the blink reflex device 100 may be configured to identify the type of degenerative neurological disorder based on an amount of change in non-stimulated blink period (e.g., between measured and baseline blink period) based on an intentional blink and/or spontaneous blink. Additionally, or alternatively, the blink reflex device 100 may be configured to sense and/or monitor eye movement (e.g., the rate and/or amount of angular rotation of the eye), pupillary response (e.g., the rate and/or amount in which the pupil of the eye changes size), and/or brain activity (e.g., electrical signals of the brain, brain waves, etc.). The blink reflex device 100 may detect potential impaired neurological function and/or the severity thereof based on a combination of changes in blink reflex and/or blink period and one or more other responses, such as changes in the subject's pupillary response, eye movement response, and/or changes in level of brain activity.

Blink reflex device 100 may be configured to detect the potential for a neurological condition in a subject based on measuring the ability of the subject to normally respond to blink-inducing stimuli and/or spontaneous blink rates. Device 100 may be configured to aid a medical practitioner and/or user to determine the integrity of the afferent sensory system entering the brainstem of the subject, the efferent motor function of the subject, as well as general homeostasis maintenance activity, such as blink in lubrication of the eye. Thus, the change in blink reflex as measured by the blink reflex device 100, may provide the user in the field a decision aid regarding whether to permit a player to return to the playing field and/or the medical practitioner insight into whether and to what extent the deep brain structures have been altered or injured due to a traumatic event to the subject.

Blink reflex device 100 and its associated methods, described herein, may enable a determination of whether a subject potentially suffers from a brain injury and/or a degenerative neurological condition. Device 100 may be configured to obtain information associated with a blink or other brain reflex, blink period, eye movement, or pupillary response of a subject. Device 100 may also, or alternatively, be configured to detect when the subject exhibits an abnormal blink (e.g., a micro-sleep, a double blink, etc.) and may reject, discard, and/or ignore any data that corresponds to an abnormal blink. Device 100 may be configured to measure the blink reflex and/or blink period for either or both eyes of the subject based on an intentional blink by the subject, a natural blink of the subject, or a reflexive blink of the subject in response to one or more different types of stimuli (e.g., mechanical, light, acoustic, electrical, or some other type of stimuli).

Blink reflex device 100 may be configured to compare information associated with a blink reflex and/or blink period obtained prior to a traumatic event experienced by the subject, with information associated with the blink reflex and/or blink period obtained after the traumatic event to identify an amount of change between the blink reflex and/or blink period before and after the trauma. Device 100 may also, or alternatively, be configured to determine whether the subject suffers from a neurological condition and/or the severity thereof based an amount of change in the blink reflex before and after the trauma relative to one or more thresholds. Additionally, or alternatively, the blink reflex device 100 may lend insight into a type of brain injury and/or specific locations in the brain that have been injured as a result of the trauma based on a respective amount of change of the blink reflex and/or blink period for each of the different types of stimulation to the subject and/or based on differences in the blink reflex between the left and right eye.

Additionally, or alternatively, the blink reflex device 100 may be configured to lend insight into a type of degenerative neurological disorder based on an amount of change in non-stimulated blink reflex before and after trauma based on an intentional blink and/or spontaneous blink without stimulation. Additionally, or alternatively, device 100 may be configured to sense and/or monitor the eye of the subject to measure the blink reflex, blink period, eye movement (e.g., the rate and/or amount of angular rotation of the eye), pupillary response (e.g., the rate and/or amount in which the pupil of the eye changes size), and/or brain activity (e.g., electrical signals of the brain, brain waves, etc.). The blink reflex device 100 may detect a neurological condition, and/or the severity thereof based on a combination of changes (e.g., before and after the subject experiences a traumatic event) in blink reflex and/or blink period relative to certain thresholds, and one or more known responses, such as changes in the subject's pupillary response, eye movement response, and/or brain activity, etc.

Blink reflex device 100 may be configured to aid a user of the blink reflex device to determine the integrity of the afferent sensory system entering the brainstem of the subject as well as the efferent motor function of the subject. Thus, the change in blink reflex as measured by the blink reflex device, may provide the user in the field a decision aid regarding whether to permit a player to return to the playing field and/or the medical practitioner insight into whether and to what extent the deep brain structures have been altered or injured due to a traumatic event to the subject.

Blink reflex device 100 may be configured to measure the blink reflex, blink period, and/or other brain reflex on an aggregate, population level to determine typical norms in development, growth, and/or aging processes and compare it to blink reflex and blink period numbers experienced by individual subjects. The metric obtained can be used to quantify deviations from population norms that will allow quantifiable measures of diagnoses that are currently described qualitatively.

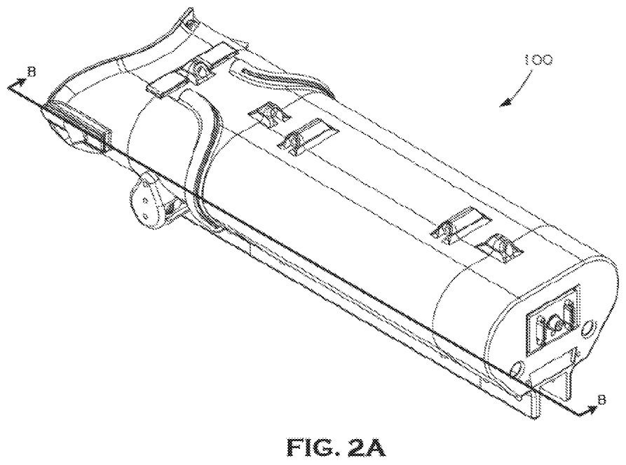

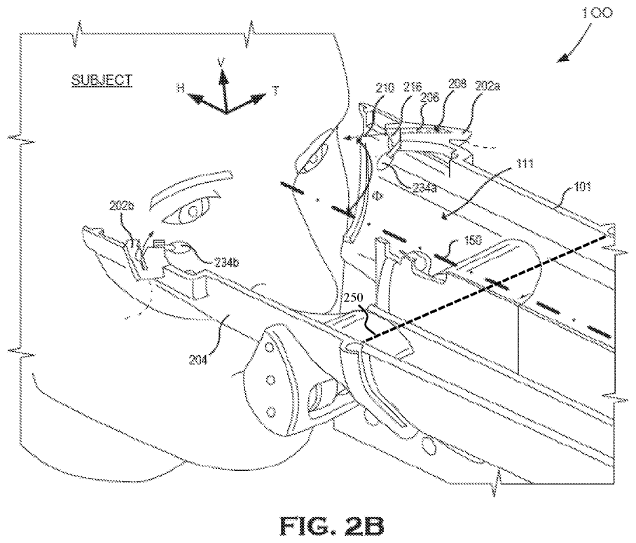

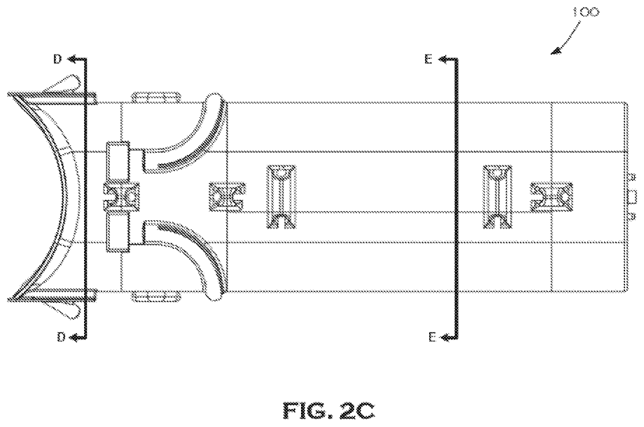

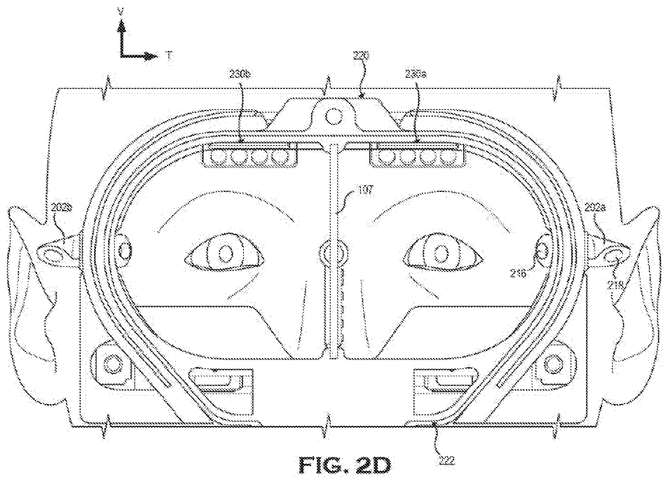

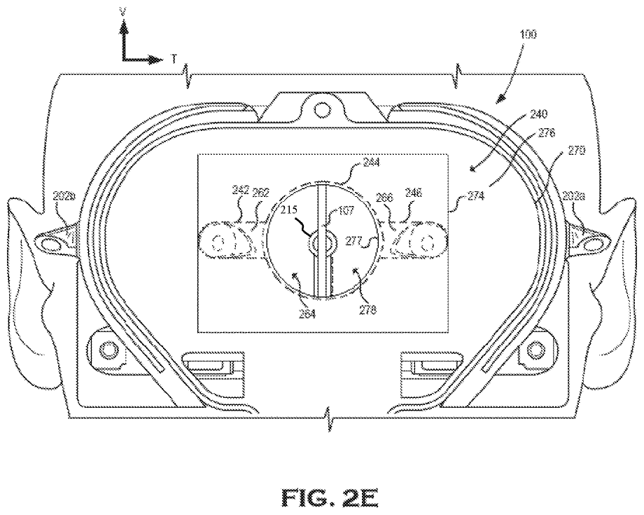

FIG. 2B is a cross sectional view of an embodiment of the brain reflex device 100 taken along line B-B of FIG. 2A. In the embodiment shown in FIG. 2B the stimulators 102 may include a first unit or first flow assembly 202a and a second unit or second flow assembly 202b that are configured to provide fluid communication between the cavity 111, defined by the housing 101, and an exterior 204 of the device 100. Fluid may be provided from the exterior 204 to the cavity 111 via a fluid pump (not shown) coupled to at least one of the flow assemblies 202a and 202b and a fluid source (not shown in the figures). For example, the fluid source may comprise an air pump or pressurized tank containing a suitable gas or a simple drop in cartridge or canister containing a suitable fluid. The flow assemblies 202a and 202b may be formed as part of the housing 101 or may otherwise be coupled to, mounted, or attached to the housing 101 so that their positioning can be adjusted if desired. The flow assemblies 202a and 202b may be positioned to stimulate each of the subject's eyes simultaneously or separately as further described herein.

The first flow assembly 202a is spaced from the second flow assembly 202b in a transverse direction T that extends parallel to a transverse axis 250. The transverse axis 250 may be substantially perpendicular to the central axis 150. The second flow assembly 202b may be configured in a substantially similar manner as the first flow assembly 202a. The position of the second flow assembly 202b on the housing 101 may be such that the second flow assembly 202b is a mirror image of the first flow assembly 202a when viewed in the horizontal direction H. The description provided below relates to the first flow assembly 202a, however, each of the features and configurations described may apply to either or both the first and second air flow assemblies 202a and 202b. It should be noted that the first flow assembly 202a may have a different configuration from the second flow assembly 202b.

The first flow assembly 202a includes an inner surface 206 that defines a channel 208 that extends through the assembly 202a so as to place the fluid source in communication with the interior cavity 111. Moreover, the channel 208 is shaped so as to direct fluid along a flow path parallel to axis 210, towards the eye at a desired angle that is optimized to elicit a blink reaction from the subject. The desired angle may include, for example, an angle at which the fluid produces an optimal blink reaction from the subject. In an aspect, the channel 208 may be positioned offset to the eye in order to provide the fluid at the desired angle. In one embodiment, the flow path terminates at the outer canthus. In another embodiment, the channel may be positioned in order to provide a fluid flow path that terminates at other facial regions, including but not limited to the temple, the medial canthus, the caruncle, the lateral canthus, or the inner canthus. In one embodiment, the channel 208 is straight, but in another embodiment shown in FIG. 2B, the channel 208 is curved so as to sweep fluid across the eye of the subject. An inner opening 216 and an outer opening 218 (See FIG. 2D) are spaced apart by the length of channel 208. In an aspect, the inner opening 216 extends in a first direction and the outer opening 218 extends in a second direction that is angularly offset from the first direction. The inner opening 216 may be located internally within the housing 101, and the outer opening 218 may be located externally to the housing 101. Outer opening 218 may include a threaded section or other attachment points to allow for connection of tubes that carry fluid from the source. Inner opening 216 may be shaped to increase or decrease the fluid flow rate or create a turbulent flow as desired to produce the optimal blink reaction. In addition, the direction of the fluid flow may be controlled by a nozzle of different shapes to direct the flow. The nozzle may be adjustable to allow fitting to a specific patient.

The first flow assembly 202a and the second flow assembly 202b may each include a microphone 234a and 234b. Each microphone 234a and 234b may be coupled to the processing unit 400 and be configured to detect a pressure variance created by fluid coming through each flow assembly 202a and 202b as the fluid flows to the eyes of the subject. If a pressure variance, such as sound, is sensed by the microphones 234a and 234b, the blink reflex device 100 will use the detection of this event to begin to track the movement of the eyelid of the subject, as further described herein. Mechanical flags (not shown) that are visible to the camera may be coupled to the blink reflex device 100 to sense/indicate fluid flow from the flow assemblies 202a and 202b.

FIG. 2D illustrates a cross section of a front view of an embodiment of the brain reflex device 100 positioned on the subject, taken along line D-D of FIG. 2C. The first flow assembly 202a and a second flow assembly 202b may be positioned in between the uppermost portion 220 of the housing 101 and lowermost portion 222 of the housing 101 in the vertical direction V. The flow assemblies 202a and 202b may be positioned on the housing 101 such that each flow assembly 202a and 202b aligns with an eye of the subject in the vertical direction V. In an alternative aspect, each flow assembly 202a and 202b may be positioned at various locations on the housing 101 in the vertical direction V so long as the flow direction 210 at the inner opening 216 is aligned to aim at least partially in the horizontal direction H towards the subject. Adjusting the position of the flow assemblies 202a and 202b enables the blink reflex device 100 to be adjusted based on a patient's anatomy.

The blink reflex device 100 may include a first set of lights 230a and a second set of lights 230b. Each set of lights 230a and 230b may be positioned within the cavity 111 towards the uppermost portion 220 of the housing 101 and aligned with one another in the transverse direction T. Each set of lights 230a and 230b may be configured to emit light at least partially in the horizontal direction H towards one or both eyes of the subject. The emitted light may create a distinct reflection pattern on each eye that may be sensed by the sensor unit 215 and used to locate each eye. Each set of lights 230a and 230b may include infrared light emitting diodes (LEDs), white light, or other light that may create a distinct reflection pattern that may be sensed by the sensor unit 215. It will be appreciated that each set of lights 230a and 230b may include a single light or may include a plurality of lights. FIG. 2E illustrates a cross section of the blink reflex device 100 taken along line E-E of FIG. 2C. An opaque plate 240 is positioned on the housing 101 such that when the blink reflex device 100 is positioned on the face of a subject (See FIG. 1D) a side (not visible) of the opaque plate 240 faces the subject in the horizontal direction H. The opaque plate 240 may be positioned at a location on the housing 101 that is furthest from the face of the subject in the horizontal direction H. In an alternative aspect, the opaque plate 240 may be positioned at a location within the housing 101 other than a location that is furthest from the face of the subject in the horizontal direction H.

The opaque plate 240 defines a first inner surface 242, a second inner surface 244, and a third inner surface 246. The first inner surface 242 extends about the horizontal direction H and defines a first opening 262. The second inner surface 244 extends circumferentially about the horizontal direction H and defines a second opening 264. The third inner surface 246 extends about the horizontal direction and defines a third opening 266. The first opening 262 opens to the second opening 264, and the second opening 264 opens to the third opening 266. In an aspect, each of the openings 262, 264, and 266 is aligned in the transverse direction T such that the second opening 264 is positioned between both of the first opening 262 and the third opening 266.

In an aspect of this disclosure, the first inner surface 242 may have a portion that extends in the transverse direction T and a portion that extends circumferentially about the horizontal direction H, thereby forming the first opening 262 into a rectangular shape with a semi-circular end. The third opening 266 may be formed substantially similarly to the first opening 262 and may form a mirror image of the first opening 262 when viewed from the horizontal direction H. In an alternative aspect, the first opening 262 may extend from an outer edge 270 of the opaque plate 240 to the second opening 264 in the transverse direction T, and the third opening 266 may extend from the second opening 264 to the outer edge 270 of the opaque plate 240 in the transverse direction T, thereby forming a continuous opening (not shown) extending through the opaque plate 240 in the transverse direction T.

The blink reflex device 100 may further include a mirror 274. The mirror 274 and the opaque plate 240 compose an eye alignment element. The mirror 274 may be coupled to a side 276 of the opaque plate 240 that opposes the side of the opaque plate 240 that faces the subject, so that the opaque plate 240 is between the mirror 274 and the subject. The mirror 274 includes an inner mirror edge 277 that defines a mirror opening 278. In an aspect, the mirror opening 278 may be configured to be consistent with the size and dimension of the second opening 264 of the opaque plate 240, such that when the mirror 274 is positioned on the opaque plate 240, the mirror opening 278 aligns with the second opening 264 in the horizontal direction H. The alignment of the mirror opening 278 with the second opening 264 may form a single opening (not labeled) that extends through both the opaque plate 240 and the mirror 274.

The mirror 274 may also be configured to cover the first opening 262 and the third opening 266 of the opaque plate 240 in the horizontal direction H. Therefore, when blink reflex device 100 is positioned on the subject, the only portion of the mirror 274 that is visible to the subject is the portion of the mirror 274 that covers the first opening 262 and the third opening 266. For example, a first line of sight may extend from a first eye of the subject to the mirror 274 in the horizontal direction H through the first opening 262, and a second line of sight may extend from a second eye of the subject to the mirror 274 in the horizontal direction H through the third opening 266.

The single opening formed by the mirror opening 278 and the second opening 264 may be configured to receive at least a portion of the sensor unit 215 within. The sensor unit 215 may be configured to monitor each eye of the subject during a blink reflex examination as further described herein.

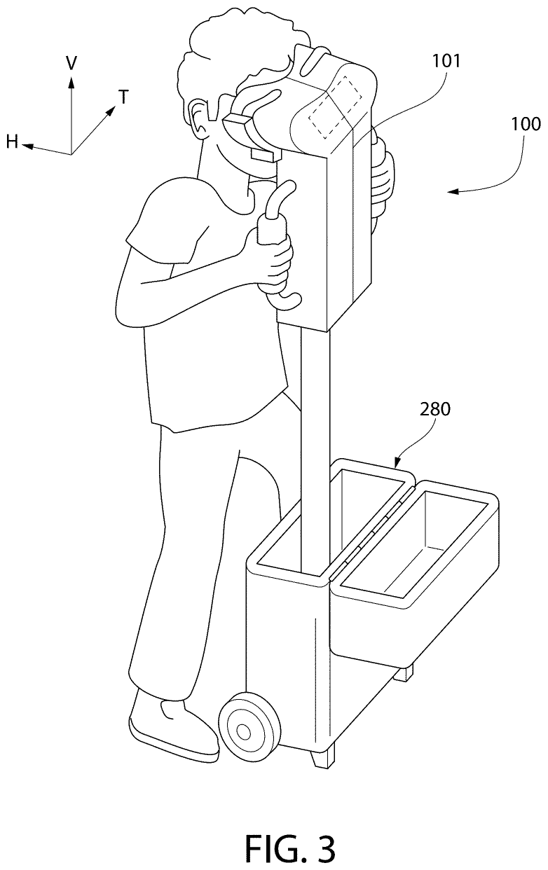

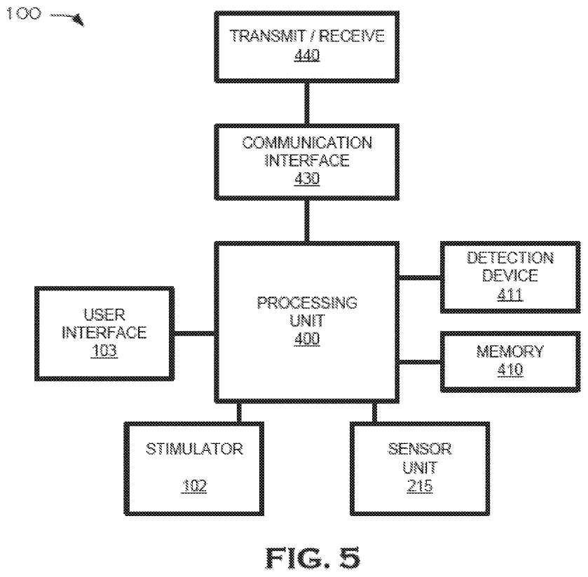

FIG. 3 illustrates an alternative configuration for the blink reflex device 100, showing a subject making use thereof. The blink reflex device 100 includes a housing unit 280. The housing unit 280 may be configured to store, for example, each of the components of the blink reflex device 100 (shown in FIG. 5) and the housing 101. The housing 101 may be configured to physically separate from the housing unit 280 while still being operatively coupled to one or more of the components stored within the unit 280. For example, the housing unit 101 may be removed from the housing unit 280 and positioned above the housing unit 280 in the vertical direction V. The housing 101 may be positioned on the subject so that the housing 101 is facing the subject in the horizontal direction H. Within the housing 101, a mirror (not visible in figures) may be positioned at a 45° angle with respect to the transverse direction T so that a line of sight of the subject is deflected towards the housing unit 280. The sensor unit 215 may be positioned within the housing unit 280 so that a line of sight of the sensor unit 215 (e.g. third line of sight) aligns with the line of sight of the subject. For example, the first line of sight, the second line of sight, and the third line of sight may all align. The sensor unit may be configured to monitor each eye of the subject facing the housing 101.

FIG. 4 is a diagram of an example environment E in which the devices and methods, described herein, may be implemented. As shown in FIG. 4, environment E may include a group of user devices 110-1, . . . , 110-J (collectively referred to herein as “user devices 110,” and individually as “user device 110”) (where J≥1) a group servers 120-1, . . . , 120-K (collectively referred to herein as “servers 120” and individually as “server 120”) (where K≥1), a blink reflex device 100 and a database 130, some or all of which are interconnected by a network 140. The number of devices and/or networks, illustrated in FIG. 4, is provided for explanatory purposes only. In practice, there may be additional networks and/or devices, fewer networks and/or devices, different networks and/or devices, or differently arranged networks and/or devices than illustrated in FIG. 4.

Also, in some implementations, one or more of the devices of environment E may perform one or more functions described as being performed by another one or more of the devices of environment E. Components of environment E may interconnect via wired connections, wireless connections, or a combination of wired and wireless connections.

User device 110 may include any computation or communication device, such as a wireless mobile communication device, that is capable of communicating with network 140. For example, user device 110 may include a radiotelephone, a personal communications system (PCS) terminal (e.g., such as a smart phone that may combine a cellular radiotelephone with data processing and data communications capabilities), a personal digital assistant (PDA) (e.g., that can include a radiotelephone, a pager, Internet/intranet access, etc.), a laptop computer, a tablet computer, a personal computer, a camera, a personal gaming system, or another type of computation or communication device.

User device 110 may further perform communication operations by sending data to or receiving data from another device, such as some other user device 110, server 120, blink reflex device 100, and/or database 130. User device 110 for example, receive an indication from blink reflex device 100 and/or server 120 that indicates whether and/or to what level of severity the subject suffers from a neurological condition. Data may refer to any type of machine-readable information having substantially any format that may be adapted for use in one or more networks and/or with one or more devices. Data may include digital information or analog information. Data may further be packetized and/or non-packetized. User device 110 may include logic for performing computations on user device 110 and may include the components illustrated in FIG. 3 in an example implementation.

Server 120 may include one or more server devices, or other types of computation or communication devices, that gather, process, search, store, and/or provide information in a manner described herein. Server 120 may communicate via network 140. Server 120 may receive from network 140 and/or blink reflex device 100 blink reflex information associated with a blink reflex of a subject (e.g., before and/or after a traumatic event to the head or spine of the subject) and may store such blink reflex information in a memory associated with server 120 and/or database 130. Server 120 may also, or alternatively, compare measured blink reflex information associated with a subject with baseline blink reflex information associated with the subject (e.g., obtained from database 130) and/or other subjects (e.g., obtained prior to a traumatic event experienced by the subject and/or other subjects and/or at a time that it was known that the subject and/or other subjects did not suffer from neurological condition to identify an amount of change between the measured blink reflex and the baseline blink reflex. Server 120 may, based on the amount of change between the measured blink reflex and the baseline blink reflex, determine whether and/or to what level of severity the subject may suffer from a brain injury and/or a degenerative neurological condition. Server 120 may provide an indication to blink reflex device 100, user device 110, or another server 120 indicating whether there are changes in a blink reflex or blink reflex parameter, and/or to what level of severity the subject potentially suffers from a brain injury and/or a degenerative neurological condition.

Blink reflex device 100 may include one or more components that are capable of obtaining, measuring, or generating certain biometric information relating to a subject and communicating with network 140. For example, blink reflex device 100 may include a radiotelephone, a personal communications system (PCS) terminal (e.g., such as a smart phone that may combine a cellular radiotelephone with data processing and data communications capabilities), a personal digital assistant (PDA) (e.g., that can include a radiotelephone, a pager, Internet/intranet access, etc.), a laptop computer, a tablet computer, a personal computer, a camera, a personal gaming system, or another type of computation or communication device. Additionally, or alternatively, blink reflex device 100 may include one or more sensor components to detect all or a portion of the subject's body (e.g., all or portions of the subject's eyes, face, head, etc.) for the purposes of measuring a blink reflex, blink period, pupillary response, eye movement, etc. associated with the subject. Blink reflex device 100 may also, or alternatively, include one or more components, to be described in greater detail that may mechanically, electrically, optically, or acoustically stimulate the subject to cause the blink reflex in the subject.

Blink reflex device 100 may obtain blink reflex information from the subject (e.g., after a traumatic event to the head and/or spine of the subject) and may compare such information to other blink reflex information (e.g., baseline blink reflex information) associated with a blink reflex of the patent and/or other subjects (e.g., prior to any trauma and/or at a time when it was known that the subject did not suffer from impaired neurological function) to determine whether the subject suffers from a neurological condition. Blink reflex device 100 may communicate with server 120, database 130 and/or user device 110, via network 140, to transmit or receive information associated with a blink reflex of the subject and/or baseline blink reflex information associated with one or more other subjects. Additionally, or alternatively, blink reflex device 100 may include logic, such as one or more processing or storage devices, that can be used to perform and/or support processing activities in connection with the operation described herein.

Database 130 may include one or more devices that store information received from blink reflex device 100, and/or server 120. For example, database 130 may store information associated with a blink reflex, blink period, eye movement, pupil response, etc. relating to one or more subject. Database 130 may also, or alternatively, store information associated with the subject (e.g., name, age, gender, race, etc.), information associated with test conditions or parameters (e.g., with or without confounding, a type of stimulation, a type of measurement, etc.), and/or information describing a type of trauma or condition (e.g., football injury, automobile accident, pre-existing condition suffered by subject, etc.).

Network 140 may include one or more wired and/or wireless networks. For example, network 140 may include a cellular network, a public land mobile network (PLMN), a second generation (2G) network, a third generation (3G) network, a fourth generation (4G) network (e.g., a long term evolution (LTE) network), a fifth generation (5G) network, and/or another network. Additionally, or alternatively, network 140 may include a wide area network (WAN), a metropolitan network (MAN), a telephone network (e.g., the Public Switched Telephone Network (PSTN)), an ad hoc network, an intranet, the Internet, a fiber optic-based network, and/or a combination of these or other types of networks.

FIG. 5 is a diagram of example components of blink reflex device 100. As shown in FIG. 5, blink reflex device 100 may include a processing unit 400, the stimulator 102, a memory 410, the sensor unit 215, the user interface 103, a detection device 411, a communication interface 430, and/or an antenna assembly 440. Although FIG. 5 shows example components of blink reflex device 100, additionally, or alternatively, blink reflex device 100 may include fewer components, additional components, different components, or differently arranged components than depicted in FIG. 5. In still other implementations, one or more components of blink reflex device 100 may perform one or more tasks described as being performed by one or more other components of blink reflex device 100.

Processing unit 400 may include a processor, a microprocessor, an application specific integrated circuit (ASIC), a field programmable gate array (FPGA), or the like. Processing unit 400 may control operation of blink reflex device 100 and its components. In one implementation, processing unit 400 may control operation of components of blink reflex device 100 in a manner similar to that described herein. For example, processing unit 400 may instruct stimulator 102 to apply a mechanical, optical, acoustic or electrical stimulation to the subject. Additionally, processing unit 400 may repeat the instruction based on a time interval, randomly (e.g., based on a random number generated by processing unit 400), and/or in response to an instruction from a user of blink reflex device 100.

Memory 410 may include a RAM, a ROM, and/or another type of memory to store data and/or instructions that may be used by processing unit 400. Memory 410 may store information associated with a blink reflex of a subject that is received from sensor unit 215, another component of blink reflex device 100 and/or network 140.

The detection device 411 is configured to determine when the stimulator 102 provides a stimulus to an eye. The detection device 411 may include, for example, the microphones 234a and 234b, or other detection or recording devices used to indicate when a stimulus is provided to an eye by the stimulator 102. The microphones 234a and 234b may record a sound of fluid as it flows from the stimulator 102.

Sensor unit 215 may include one or more components to detect, measure, scan, and/or record all or a portion of a body of a subject, such as, for example, the face, the eyes, a portion of one or both of the eyes (e.g., eyelid, a pupil, etc.), etc. For example, sensor unit 215 may include one or more cameras, photodiodes, electro-optical sensors, infrared sensors, ultraviolet sensors, laser diode sensors, electrodes, focal plan arrays (FPA), antenna, etc. to detect, measure, scan, and/or record the subject (e.g., the eye, eyelid, face, etc. of the subject) in one or more portions of the electromagnetic spectrum (e.g., ultraviolet, visual, thermal, far infrared, microwave, electrical, x-ray, etc.). Sensor unit 215 may include a field of view, directivity, scan rate (e.g., scans per minute, per second, etc.), pixel density (e.g., pixels per line or array), spectral range, dynamic range, level of resolution (e.g., dots per inch), a frame rate, a shutter speed, gain control, etc. that enables the eye, eyelid, eyelashes, etc. of the subject to be detected and tracked as a function of time before, during, and after stimulation is applied and/or the subject intentionally or unintentionally blinks. In one example, sensor unit 215 may measure information associated with a blink reflex of the subject and may provide such information to processing unit 400. Additionally, or alternatively, sensor unit 215 may measure other information associated with eye movement, pupillary response, brain waves, etc. associated with the subject and may provide such other information to processing unit 400.

User interface 103 may include one or more components that enable information to be input to the blink reflex device 100 and/or for outputting information from the blink reflex device 100. For example, user interface 103 may include buttons, a touch screen, control buttons, a keyboard, a pointing device, etc. to enable a user, of blink reflex device 100, to input information associated with a measurement (e.g., type and/or magnitude of stimuli; selection of right, left or both eyes, retrieval of information associated with baseline blink reflex, to power up, to power down, etc.) and/or to permit data and control commands (e.g., on, off, record, play, etc.) to be input into blink reflex device 100 via user interface 103. User interface 103 may also, or alternatively, render video, images, audio, graphical, or textual information associated with a blink reflex of the subject for display to enable the subject or medical practitioner to determine whether the subject potentially suffers from a neurological condition or the severity thereof

Communication interface 430 may, for example, include one or more components to enable blink reflex device 100 to communicate with network 140 via transmit/receive 440. For example, communication interface 430 may include a transmitter that converts baseband signals from processing unit 400 to signals (e.g., microwave signals, infrared signals, etc.) that can be transmitted, via transmit/receive 440 to network 140. Communication interface 430 may also, or alternatively, include a receiver that converts signals received from transmit/receive 440 to baseband electrical or optical signals that can be processed by processing unit 400. Additionally, or alternatively, communication interface 430 may include a transceiver to perform functions of both a transmitter and a receiver of wireless communications (e.g., radio frequency, infrared, visual optics, etc.), wired communications (e.g., conductive wire, twisted pair cable, coaxial cable, transmission line, fiber optic cable, waveguide, etc.), or a combination of wireless and wired communications.

Transmit/receive 440 may include one or more antennas to transmit and/or receive radio frequency (RF) signals over the air. Transmit/receive 440 may, for example, receive RF signals from communication interface 430 and transmit them over the air, and receive RF signals over the air and provide them to communication interface 430. Additionally, or alternatively, transmit/receive 440 may include one or more optical devices to transmit and/or receive optical signals (e.g., visual, infrared, laser, ultraviolet, etc.) over the air. Transmit/receive 440 may, for example, receive optical signals from communication interface 430 and transmit them over the air, and/or receive optical signals over the air and provide them to communication interface 430.

As described in detail below, blink reflex device 100 may perform certain operations described herein in response to processing unit 400 executing software instructions of an application contained in a computer-readable medium, such as memory 410. The software instructions may be read into memory 410 from another computer-readable medium or from another device via communication interface 430. The software instructions contained in memory 410 may cause processing unit 400 to perform processes that will be described later. Alternatively, hardwired circuitry may be used in place of or in combination with software instructions to implement processes described herein. Thus, implementations described herein are not limited to any specific combination of hardware circuitry and software.

As will be described in detail below, device 100 may perform certain operations relating to video content ingestion. Device 100 may perform these operations in response to the processing unit 400 executing software instructions contained in a computer-readable medium, such as memory 410. A computer-readable medium may be defined as a non-transitory memory device. A memory device may include space within a single physical memory device or spread across multiple physical memory devices. The software instructions may be read into memory 410 from another computer-readable medium or from another device. The software instructions contained in memory 410 may cause the processing unit 400 to perform processes described herein. Alternatively, hardwired circuitry may be used in place of or in combination with software instructions to implement processes described herein. Thus, implementations described herein are not limited to any specific combination of hardware circuitry and software.

The stimulator 102 may, for example, include a component that outputs a mechanical stimulation, such as, for example, a puff of fluid at a predetermined pressure, direction, quantity, velocity, duration, etc. The term fluid, as used herein, includes a gas, liquid, or any material the flows or behaves in a like manner (e.g., nitrogen, air, water, water vapor, etc.). The puff of fluid may be directed to one or both eyes of the subject or within proximity of the eye and/or eyelid (e.g., within one-quarter inch, on-half inch, one inch, one and one-half inch, etc. of the eye, eyelid, eyelashes, etc.) to cause the subject to exhibit a blink reflex. The stimulator 102 may also, or alternatively, include a component that applies a controlled mechanical pressure to the proximity of the eye and/or eyelid (e.g., a pin prick, a pinch, etc.).

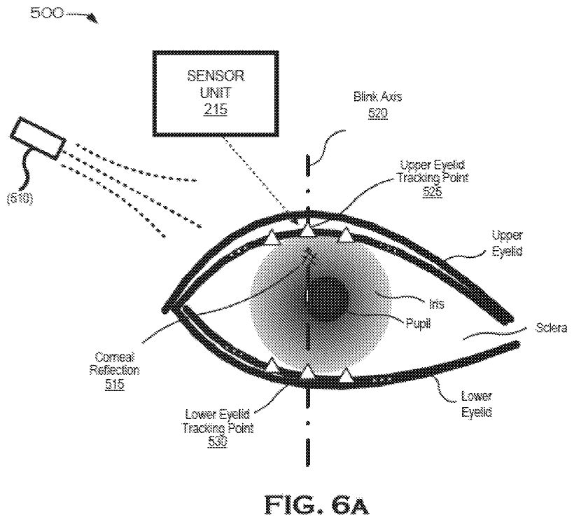

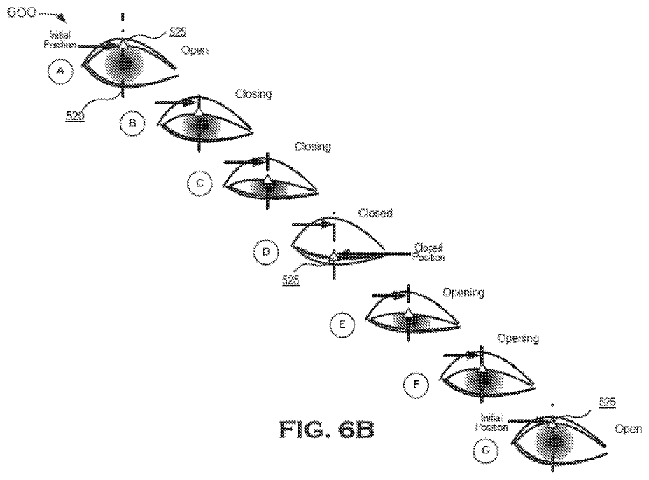

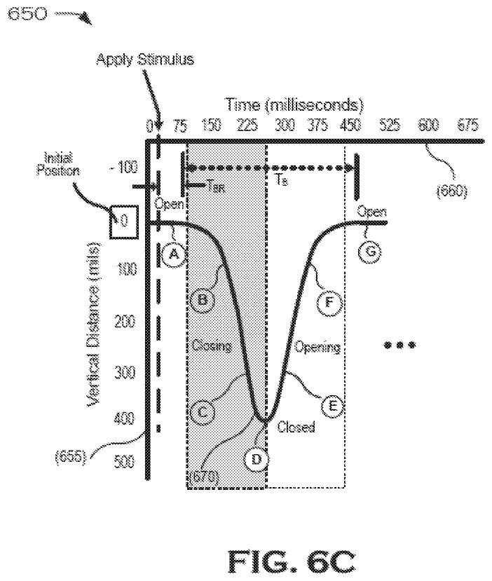

FIG. 6A is a diagram of an example eyelid tracking scheme 500 (hereinafter, “tracking scheme 500”) associated with a subject. In an example implementation, tracking scheme may be used by blink reflex device 100 to perform an operation to determine a blink reflex and/or a blink period of a subject by measuring, as a function of time, the location of all or a portion of one or both eyelids when the subject initiates a blink, performs the blink, and/or completes the blink. As shown in FIG. 6A, tracking scheme 500 may include light source 510, a corneal reflection 515, a blink axis 520, an upper eyelid tracking point 525, and a lower eyelid tracking point 530. Light source 510 may include a light bulb, an LED, a low power laser that does not cause damage to the eye (e.g., less than approximately 5 milliwatts (mW), etc.) that emits light that can be directed to an eye of the subject. Light source 510 may also, or alternatively, be associated with stimulator 102 and/or some other component of blink reflex device 100.

By way of example, light source 510 may emit a beam of light (e.g., as shown by the dotted line between light source 510 and the iris of the eye in FIG. 6A) in a manner that is incident on the cornea portion (e.g., a membrane that covers the iris and pupil portions of the eye) of the eye of a subject. The beam of light may enter the cornea and/or may reflect off the cornea and/or iris portion of the eye to cause a reflection of light to appear on a portion of the surface of the eye (e.g., shown by the “#” labeled corneal reflection 515). Sensor unit 215 may detect corneal reflection 515 and may identify a first point along an approximately vertical blink axis 520 (e.g., shown as the alternating dashed and dotted vertical line labeled “blink axis 520”) at which the upper eyelid intersects blink axis 520 (e.g., shown as a “A” labeled “upper eyelid tracking point 525” in FIG. 6A). Additionally, or alternatively, sensor unit 215 may detect corneal reflection 515 and may identify a second point along blink axis 520 at which the lower eyelid intersects blink axis 520 (e.g., shown as an “A” labeled “lower eyelid tracking point 530” in FIG. 6A).

Additionally, or alternatively, sensor unit 215 may monitor and/or track the movement of the upper eyelid (e.g., before, during, and/or after the subject blinks) based on the upper eyelid tracking point 525 and/or the lower eyelid tracking point 530. Sensor unit 215 may also, or alternatively, identify one or more different upper eyelid tracking points 525 associated with the upper eyelid (e.g., shown by the other “Δs” located on the upper eyelid of FIG. 6A) and may monitor and/or track the vertical position of one, some, or all of the different upper eyelid tracking points 525 (e.g., based on each individual vertical position, a sum of the vertical positions, an average of the vertical positions, etc. of the different upper eyelid tracking points 525). Sensor unit 215 may also, or alternatively, monitor and/or track the vertical position of one, some, or all of the different lower eyelid tracking points 530 in a manner similar to that described above.

Additionally, or alternatively, sensor unit 215 may track upper eyelid tracking points 525 and/or lower eyelid tracking points 530 in a generally horizontal direction that is approximately orthogonal to blink axis 520. Additionally, or alternatively, sensor unit 215 may identify a tracking point that enables the movement of the eye to be tracked, for example, in the vertical direction, the horizontal direction, or some other direction. In this example, sensor unit 215 may track the change in location of corneal reflection 515 to determine eye movement. Additionally, or alternatively, sensor unit may identify some other tracking point, associated with the eye or portion thereof (e.g., an edge of the iris, the pupil, etc.).

FIG. 6B is a diagram of example stages 600 of a blink of an eye of a subject from which a blink reflex or blink period can be measured. As shown in FIG. 6B, eye blink stages 600 may include a collection of eye blink stages A through G associated with the blink of the eye of the subject. The number of eye blink stages of FIG. 6B is provided for explanatory purposes. In practice, there may be additional stages, fewer stages, or different stages than are shown in FIG. 6B. While stages 600 is described in the context of upper eyelid tracking point 525, associated with the upper eyelid of the subject, additionally, or alternatively, stages 600 may be described in the context of one or more different upper eyelid tracking points 525 and/or one or more lower eyelid tracking points 530 associated with the lower eyelid of the subject.

Eye blink stage A may correspond to a first state of the eye of the subject at a first time prior to the initiation of a blink. During eye blink stage A, the eye may be open and/or the location of upper eyelid tracking point 525 may correspond to an initial position (e.g., shown as the righting pointing arrow labeled “Initial Position” in FIG. 6B, also referred to as “tonic lid position”) on the approximately vertical blink axis 520 with which upper eyelid tracking point 525 coincides. Eye blink stage B may correspond to a second state of the eye at a second time after the initiation of a blink when the upper eyelid and/or lower eyelid begins to close. During eye blink stage B, the eye may begin closing and/or the location of upper eyelid tracking point 525 may correspond to a first position on the vertical axis that is located below the initial position on the vertical axis. Eye blink stage C may correspond to a third state of the eye of the subject at a third time that occurs after the second time. During eye blink stage C, the eye may be continuing to close and/or the location of upper eyelid tracking point 525 may correspond to a second position on blink axis 520 that is located below the first position. Eye blink stage D may correspond to a fourth state of the eye of the subject at a fourth time that occurs after the third time. During eye blink stage D, the eye may be closed and/or the location of upper eyelid tracking point 525 may correspond to a third position (e.g., shown as the “closed position” in FIG. 6B) on blink axis 520 that is located below the second position. In implementations in which lower eyelid tracking point 530 (not shown in FIG. 6B) is being monitored and/or tracked by blink reflex device 100, upper eyelid tracking point 525 and lower eyelid tracking point may be located at approximately the same position on blink axis 520.

Eye blink stage E may correspond to a fifth state of the eye of the subject at a fifth time that occurs after the fourth time. During eye blink stage E, the eye may begin opening and/or the location of upper eyelid tracking point 525 may correspond to a fourth position on blink axis 520 that is located above the third position. Eye blink stage F may correspond to a sixth state of the eye of the subject at a sixth time that occurs after the fifth time. During eye blink stage F, the eye may continue opening and/or the location of upper eyelid tracking point 525 may correspond to a fifth position on blink axis 520 that is located above the fourth position. Eye blink stage G may correspond to a sixth state of the eye of the subject at a sixth time that occurs after the fifth time. During eye blink stage G, the eye may be open and/or the location of upper eyelid tracking point 525 may correspond to a sixth position on blink axis 520 that is located above the fifth position. Additionally, or alternatively, the sixth position may coincide approximately with the location of the initial position of eye blink stage A. If this location is deemed significantly different than the initial position of the eye, it may also be an additional indicator that there is altered brain function suggestive of brain injury by comparison with known prior baselines in the database for the subject.

FIG. 6C is a diagram of an example blink reflex response 650 (hereinafter, “response 650”) associated with a blink reflex and blink period of a subject. Response 650 may be measured and/or created by blink device 100 based on a blink reflex and/or blink period associated with an eye of a subject. As shown in FIG. 6C, response 650 may include a distance scale 655, a time scale 660, and a blink function 670 (hereinafter, “blink function 670”). Distance scale 655 may include a range of distance (e.g., shown as the vertical axis labeled “Vertical Distance (mils)” ranging from −100 mils to +500 mils) that the upper eyelid tracking point 525, lower eyelid tracking point 530, or a combination of upper and lower eyelid tracking points 525 and 530, respectively, travel relative to an initial position on blink axis 520 when the subject blinks. Time scale 655 may include a range of time (e.g., shown as the horizontal axis labeled “Time (milliseconds)” ranging from 0 to 675 ms or some other period of time) during which the eye of the subject blinks one or more times. Blink function 670 may represent a relationship between a vertical distance that the eyelid travels (e.g., upper eyelid tracking point 525, lower eyelid tracking point 530 or some combination thereof) as shown on distance scale 655 as a function of time on time scale 660 when the subject blinks. The vertical dashed line labeled “Apply Stimulus” may identify a time (e.g., based on time scale 655) at which stimulation is applied to the subject.

Blink reflex device 100 may measure the blink reflex of a subject and may create blink function 670 based on the distance traveled by one or both eyelids of the subject as a function of time. For example, blink reflex device 100 may, in a manner similar to that described with respect to FIG. 6B, begin to track an eyelid tracking point (e.g., upper eyelid tracking point 525, lower eyelid tracking point 530 and/or some combination thereof) of the subject (e.g., at T=0 on time scale 660) and may apply a stimulus to the subject (e.g., with a puff of fluid, a mechanical, acoustic, electrical optical etc. stimulus). Blink reflex device 100 may track the movement of the eyelid tracking point and may identify a time at which eyelid tracking point begins to move vertically relative to blink axis 520 and/or a blink is initiated by the subject in response to the stimulus. Blink reflex device 100 may determine a time period from the time when the stimulus is applied to when the eyelid tracking point begins to move or the blink is initiated (“individual latency”). The time period may correspond to the blink reflex (e.g., shown as TBR in FIG. 6C).

Additionally, or alternatively, blink reflex device 100 may measure the blink period associated with the phases of the blink, the aggregate curve referred to as the morphology of the blink. For example, when the eye of the subject is in the open state (e.g., stage A of FIG. 6B), blink reflex device 100 may determine that the eyelid (e.g., upper eyelid tracking point 525, lower eyelid tracking point 530, or some combination thereof) is located at the initial position on distance scale 655 (e.g., approximately 0 mils) as shown by blink function 670 (e.g., shown as prior to 75 ms on time scale 660). When the eye of the subject is closing (e.g., stages B and C of FIG. 6B), blink reflex device 100 may determine that the tracking point of the eyelid has changed to a different position relative to the initial position (e.g., 50, 100, 150, 250, 350, etc. mils on distance scale 655) as shown by blink function 670 (e.g., shown as between approximately 75 and 250 ms on time scale 660). When the eye of the subject is in the closed state (e.g., stage D of FIG. 6B), blink reflex device 100 may determine that the eyelid tracking point is located a greatest distance from the initial position on distance scale 655 (e.g., shown as approximately 400 mils) as shown by blink function 670 (e.g., shown as between approximately 251 and 275 ms on time scale 660).

Additionally, or alternatively, when the eye of the subject is opening (e.g., stages E and F of FIG. 6B), blink reflex device 100 may determine that the tracking point of the eyelid has changed to a different position relative to the initial position (e.g., 50, 100, 150, 250, 350, etc. mils on distance scale 655) as shown by blink function 670 (e.g., shown as between approximately 275 and 450 ms on time scale 660). When the eye of the subject has returned to the open state (e.g., stage G of FIG. 6B), blink reflex device 100 may determine that the eyelid (e.g., upper eyelid tracking point 525, lower eyelid tracking point 530, or some combination thereof) has returned to the approximate initial position on distance scale 655 (e.g., approximately 0 mils) as shown by blink function 670 (e.g., shown as after 450 ms on time scale 660).

Blink reflex device 100 may determine a time period (the blink period or sometimes referred to as “blink duration”) for the eye lid to travel from the initial position, to the closed position and return to the initial position (e.g., shown as TB in FIG. 6C).

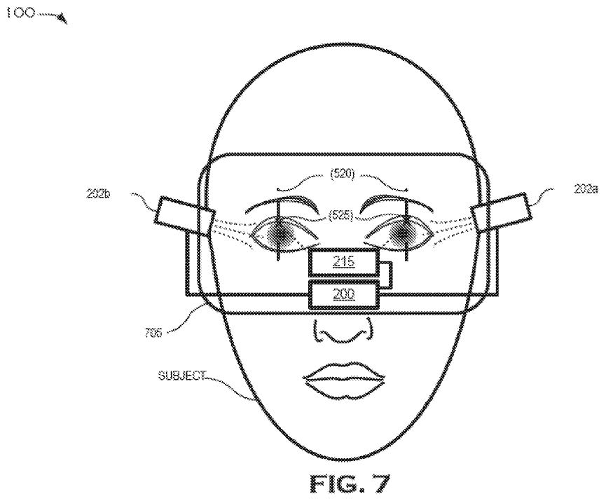

FIG. 7 illustrates an example of the blink reflex device 100. As shown in FIG. 7, of the blink reflex device 100 may include a housing 101 and one or more components described above with respect to FIG. 5 including processing unit 400, stimulator 102, and sensor 215. Housing 101 may include a material of sufficient strength, structure, and/or rigidity to enable some or all of the components, described above with respect to FIG. 5, to be attached and to operate in order to measure a blink reflex associated with a subject. Housing 101 may also, or alternatively, have a shape that corresponds to a subject's face so as to securely cover one or both eyes of the subject (e.g., similar to a scuba mask, goggles, etc.), worn by the subject, or in which all or a portion of the head of the subject can be inserted in a manner that enables stimulator 102 and/or sensor 215 sufficient line of sight to the eye or proximity thereof. While other components described with respect to FIG. 5, including memory 410, user interface 103, communication interface 430 and transmit/receive 440, are not shown in FIG. 7 for simplicity, in practice, blink reflex device 100 may include one or more such components of FIG. 5, and/or additional components, fewer components, different components or differently arranged components than are described with respect to FIG. 7.

As shown in FIG. 7, blink reflex device 100 may include housing 101, stimulator 102, sensor 215, processing unit 400 and one or more other components described above with respect to FIG. 5 (not shown in FIG. 7). Stimulator 102 may include one or more mechanical modules, such as the first and second flow assemblies 202a and 202b. The first flow assembly 202a may be associated with the right eye of the subject and the second flow assembly 202b may be associated with the left eye of the subject. One or both of the flow assemblies 202a and 202b may output a puff of fluid (e.g., air, nitrogen, water, water vapor, etc.) in the direction of one or both eyes of the subject. The puff of fluid may make contact with one or both eyes of the subject under sufficient velocity and/or pressure in a manner that causes a blink reflex in the subject that can be detected and measured by sensor 215 in a manner similar to that described above with respect to FIGS. 6A and 6B (e.g., by tracking the movement of upper eyelid tracking point 525 and/or lower eyelid tracking point 530 (not shown in FIG. 7). The first and second flow assemblies 202a and 202b may also, or alternatively, be installed in and/or attached to housing 101. Additionally, or alternatively, the air flow assemblies 202a and 202b may output the puff of air based on an instruction received from processing unit 400 and/or may output a signal to processing unit 400 indicating that the puff of air has been output by at least one of the first and second air flow assemblies 202a and 202b.

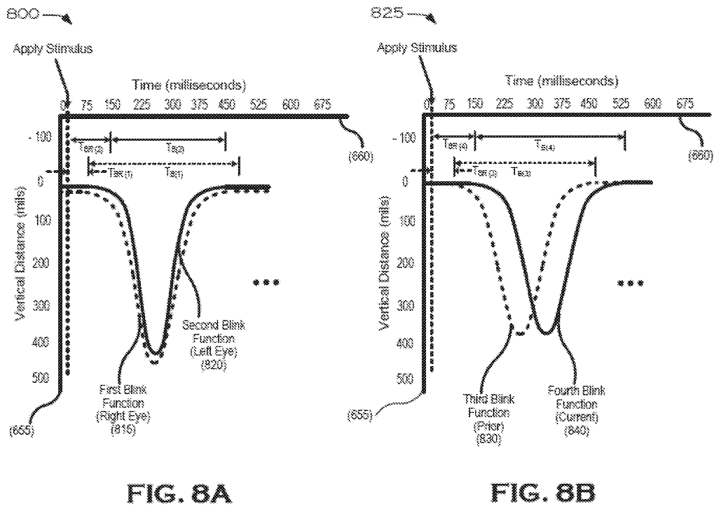

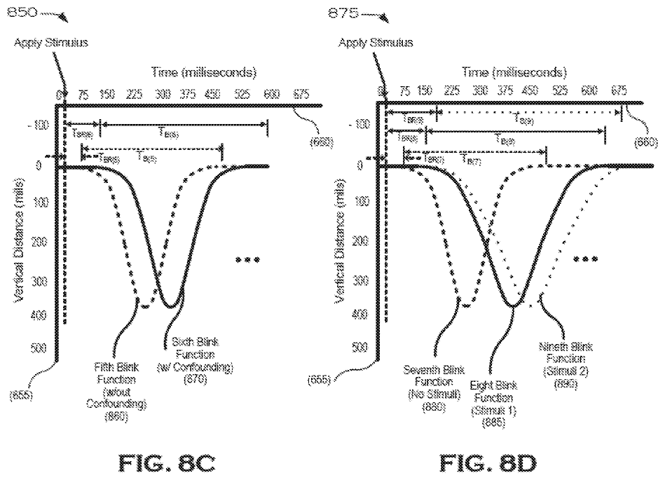

FIGS. 8A-8D are diagrams of different types of example blink reflex responses 800-875, respectively, associated with a subject. Blink reflex responses 800-875 may be obtained, measured and/or generated by blink reflex device 100 and/or blink reflex devices 100-100 based on a blink reflex of a subject. As shown in FIGS. 8A-8D, blink reflex responses 800-875, respectively, may each include distance scale 655 and time scale 660 as described above with respect to FIG. 6C. Blink reflex responses 800-875 are shown in FIGS. 8A-8D, respectively, as corresponding to a single blink of the eye that occurs during a particular time period, for simplicity. In practice, blink reflex responses 800-875 may correspond to two or more blinks of the eye that occur over an extended period of time that is greater than that the particular time period.

As shown in FIG. 8A, blink reflex response 800 (hereinafter “response 800”) may include a first blink function 815 associated with the right eye and a second blink function 820 associated with the left eye. First blink functions 815 and second blink function 820 may, in a manner similar to that described above with respect to FIG. 6C, represent a blink reflex of the right eye and left eye of the subject, respectively, obtained by blink reflex device 100. Blink reflex device 100 may determine a first time period, associated with first blink function 815 that corresponds to a first blink reflex of the right eye (e.g., T_{BR(1)}). Blink reflex device 100 may determine a second time period, associated with second blink function 820 that corresponds to a second blink reflex of the left eye (e.g., T_{BR(2)}).

Additionally, or alternatively, blink reflex device 100 may apply stimulus (e.g., mechanical, optical, acoustic, electrical, etc.) to one eye and/or the proximity thereof (e.g., the right eye) and may obtain a first blink reflex from the right eye (e.g., T_{BR(1)}) and a first blink reflex of the left eye (e.g., T_{BR(2)}) in response to the stimulus to the right eye. The right eye to which the stimulus is applied may sometimes be referred to herein as the “stimulated eye” or the “ipsilateral eye.” The left eye, that did not receive the stimulus, may sometimes be referred to herein as the “non-stimulated eye” or the “contralateral eye.” In such a case, there may be a period of delay between the initiations of the blink reflex of the ipsilateral eye relative to the other, contralateral eye. The period of delay may correspond to the difference in blink reflex between the ipsilateral eye and contralateral eye (e.g., T_{BR(1)}<T_{BR(2)}). Such a difference in blink reflex, between the ipsilateral eye and the contralateral eye, may be due to the additional neural pathways and/or distance that electrical brain signals must travel to trigger the blink reflex in the non-stimulated, contralateral eye (e.g., the left eye). In the event that the difference in blink reflex between the ipsilateral eye and contralateral eye (e.g., ΔT_{BR}=T_{BR(1)}−T_{BR(2)}) changes by more than a first threshold (e.g., after a traumatic event to the head or spine of the subject), such neural pathways may have been effected or impaired by the trauma or some neurological functional impairment.

Additionally, or alternatively, despite the larger blink reflex of the non-stimulated eye (e.g., the left eye), the first blink period of the non-stimulated eye (e.g., T_{B(2)}) may be less than the first blink period of the stimulated right eye (e.g., T_{B(2)}<T_{B(1)}, where T_{B(1)} is the first blink period of the right eye). In the event that the difference in blink period between the ipsilateral eye and contralateral eye (e.g., ΔT_{B}=|T_{B(1)}−T_{B(2)}|) changes by more than a second threshold (e.g., after a traumatic event to the head or spine of the subject), such neural pathways may have been effected or impaired by the trauma or some neurological functional impairment.

If, however, blink reflex device 100 applies stimuli to both eyes (e.g., either sequentially or at approximately the same time), the difference between the right eye blink reflex or blink period and the left eye blink reflex or blink period, respectively, may be an indication of a brain injury and/or a degenerative neurological condition associated with one or both sides of the brain and/or one or more neural pathways of the brain through which electrical brain signals that trigger the blink reflex travel.

As shown in FIG. 8B, blink reflex response 825 (hereinafter “response 825”) may include a third blink function 830 and a fourth blink function 840. Third blink function 830 may, in a manner similar to that described above with respect to FIGS. 6C and 8A, represent a third blink reflex of an eye of the subject obtained, by blink reflex device 100, at a third point in time before the subject suffered from trauma or was known not to be suffering from a brain injury or degenerative neurological disorder) (sometimes referred to herein after “baseline blink reflex”). Additionally, or alternatively, third blink function 830 may represent a combination of third blink reflex functions (e.g., an average, a mean, a median, a weighted average, etc.) associated with one or more other subjects that are known not to suffer from a brain injury or degenerative neurological disorder. Such other subjects may, for example, be associated with one or more similar demographic parameters relative to the subject (e.g., similar age group, gender, race, etc.). Fourth blink function 840 may correspond to a fourth blink reflex of the eye obtained, by blink reflex device 100, at a fourth point in time that occurs at a current time and/or within a short time period after the subject is known to have suffered from trauma (e.g., within 5 minutes, 10 minutes, 30 minutes, 1 hour, 2 hours, 5 hours, 12 hours, 24 hours, etc.). Blink reflex device 100 may, based on third blink function 830, determine a third blink reflex of the eye (e.g., T_{BR(3)}) and/or, based on fourth blink function 840, determine a fourth blink reflex of the eye (e.g., T_{BR(4)}). In the event that the difference in baseline blink reflex and the post-trauma blink reflex (e.g., ΔT_{BR}=T_{BR(3)}−T_{BR(4)}) changes by more than a third threshold, a potential brain injury or degenerative neurological condition may exists within the subject. Similarly, in the event that the difference between baseline blink period and the post-trauma blink period (e.g., (e.g., ΔT_{B}=|T_{BR(3)}−T_{BR(4)}) is greater than a fourth threshold, a potential brain injury or degenerative neurological condition may exist within the subject.

As shown in FIG. 8C, blink reflex response 860 (hereinafter “response 860”) may include a fifth blink function 860 and a sixth blink function 870. Fifth blink function 860 may identify a fifth blink reflex and/or fifth blink period of an eye (e.g., the right or left eye) of the subject obtained without confounding the subject prior to and/or while applying stimuli to the subject. Sixth blink function 870 may identify a sixth blink reflex and/or a sixth blink period of the eye (e.g., the right or left eye) based on confounding the subject prior to and/or while applying the stimuli to the subject. As shown with respect to fifth blink function 860, blink reflex device 100 may determine the fifth blink reflex (e.g. T_{BR(5)}) and/or the fifth blink period (e.g., T_{B(5)}) of the eye without confounding. As shown with respect to sixth blink function 870, blink reflex device 100 may determine the sixth blink reflex (e.g., T_{BR(6)}) and/or the sixth blink period (e.g., Two) of the eye with confounding. Blink reflex device 100 may, in a manner to be described later, use differences between the fifth and sixth blink reflexes with and without confounding and/or differences in the fifth and sixth blink periods with and without confounding to determine whether a potential brain injury or degenerative neurological condition exists within the subject.

As shown in FIG. 8D, blink reflex response 875 (hereinafter “response 875”) may include a seventh blink function 880, an eighth blink function 885, and a ninth blink function 890 obtained and/or created by blink reflex device 100. Seventh blink function 880 may identify a seventh blink reflex (e.g., T_{BR(7)}) and a seventh blink period (e.g., T_{B(7)}) of the eye of the subject (e.g., right or left eye) without providing any stimuli to the subject. Eighth blink function 885 may identify an eighth blink reflex (e.g., T_{BR(8)}) and an eighth blink period (e.g., T_{B(8)}) of the eye based on providing a first type of stimuli (e.g., mechanical, optical, acoustic, electrical, etc. stimuli) to the eye or proximity thereof of the subject. Ninth blink function 890 may identify a ninth blink reflex (e.g., T_{BR(9)}) and a ninth blink period (e.g., T_{B(9)}) of the eye based on applying a second, different type of stimuli to the subject. Blink reflex device 100 may, in a manner to be described below, use the differences in one or more of these blink reflex and between these blink periods to determine whether a potential brain injury or degenerative neurological condition exists within the subject.

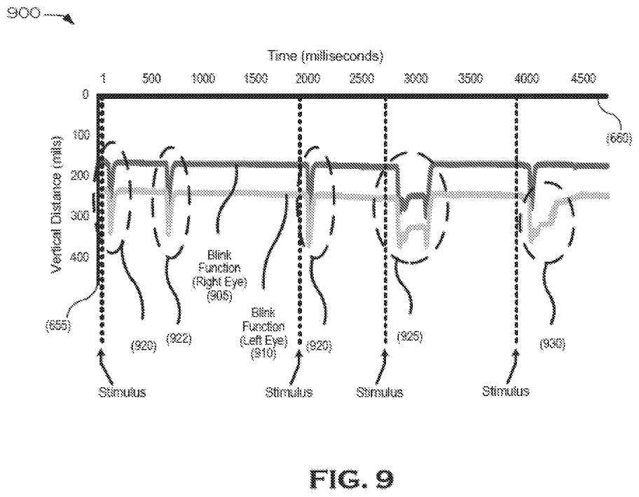

FIG. 9 is a diagram of an example blink reflex response 900 (hereinafter, “response 900”), associated with a subject that includes data to be removed and/or filtered from response 900. As shown in FIG. 9, response 900 may be created by blink reflex device 100 and/or blink reflex devices 100-100, based on multiple blinks of a right eye of a subject, associated with blink function 905 and multiple blinks of a left eye of the subject associated with blink function 910. In a manner similar to that described above with respect to FIGS. 8A-8D, a normal blink of the right and/or left eye may correspond to approximately symmetric peaks in portions of blink responses 905 and/or 910 (e.g., as shown by ellipses 920). Such peaks may correspond to a normal blink reflex in response to stimulus being provided to the subject and/or a normal blink period based on stages A through G (FIG. 6B) in which the eye begins in the open state (e.g., stage A in which the eyelids are in an initial position), transitions to the closed state (e.g., stage D) and returns to the open state (e.g., stage G in which the eyelids return to approximately the initial position of stage A). Additionally, blink responses 905 and/or 910 may include a normal blink reflex and/or blink period that is voluntary or spontaneous that is not in response to any stimulus being provided to the subject (e.g., as shown by ellipse 922).

Additionally, or alternatively, blink reflex device 100 may detect a blink that is not a normal blink (sometimes referred to as a “double blink”) in which one or both eyes transition from the open state to the close state and begin returning to the open state, but reverse direction and begin closing and/or returning to the closed state prior to reaching the open state (e.g., as shown by ellipse 925). Additionally, or alternatively, blink reflex device 100 may detect a blink that is not a normal blink (sometimes referred to as a “micro-sleep”) in which one or both eyes transition from the open state to the close state and begin returning to the open state at rate that is substantially slower than that associated with a normal blink. Such a double blink and/or micro-sleep event may be an indication that the subject is experiencing fatigue and/or may occur over a prolonged period that is substantially longer than the normal blink reflex (e.g., 5 times longer, 10 times longer, 20 times longer). Such data could be used, by blink reflex device 100 to identify potential impairments in cognitive alertness of the subject and/or to determine whether a potential brain injury or degenerative neurological condition exists within the subject. Additionally, or alternatively, for determining a blink reflex and/or blink period, data associated with a double blink and/or micro-sleep event may introduce errors into the determination of the period of time during which a blink reflex occurs. Blink reflex device 100 may reject, discard, or ignore such data when determining the blink reflex and/or blink period.

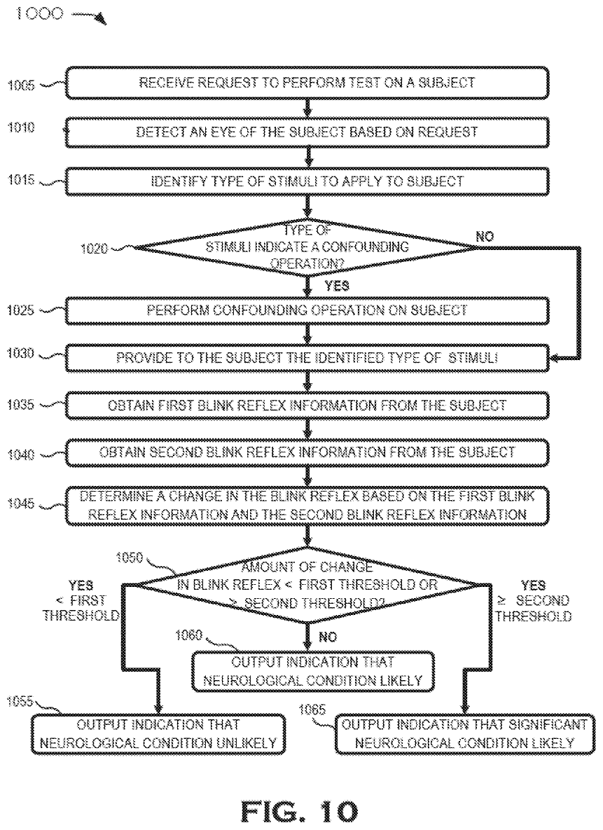

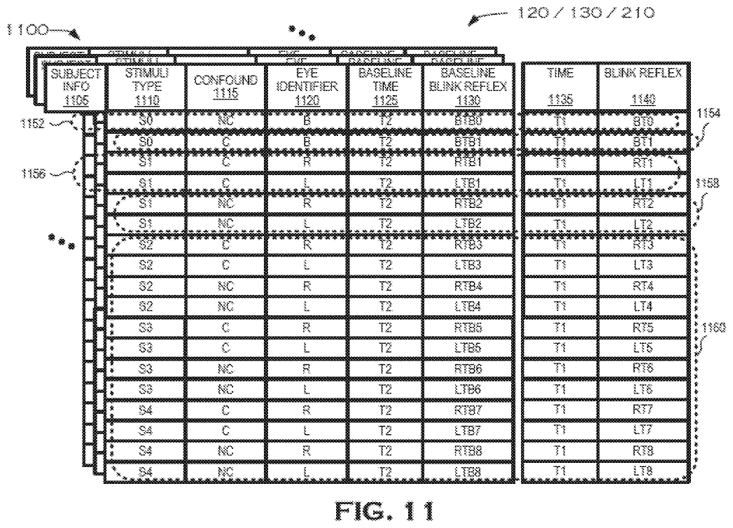

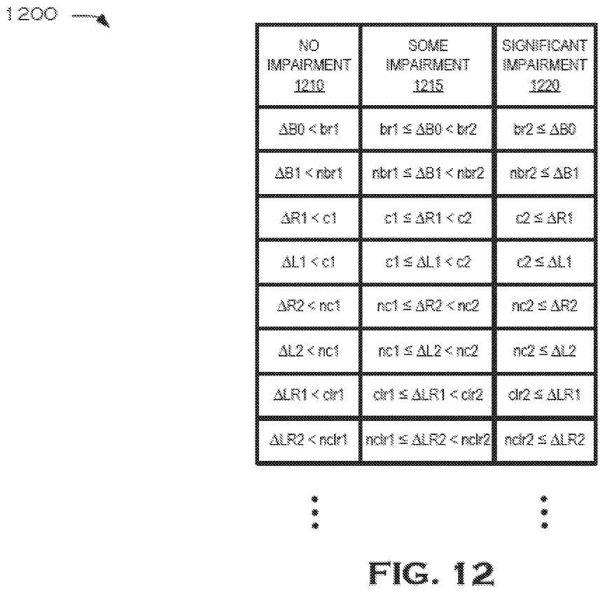

FIG. 10 is a flowchart of an example process 1000 for determining whether a subject suffers from brain injury or a degenerative neurological condition. Process 1000 may be performed by one or more devices associated with blink reflex device 100 and/or 100-100. Additionally, or alternatively, some or all of process 1000 may be performed by a device, or collection of devices separate from, or in combination with blink reflex device 100 and/or 100-100. FIG. 11 is a diagram of an example data structure 1100 that may store information associated with a blink reflex of a subject. FIG. 12 is a diagram of an example data structure 1200 that stores information associated with a change in blink reflex of a subject. Process 1000 of FIG. 10 will be described with references to all or a portion of data structure 1100 of FIG. 11 and data structure 1200 of FIG. 12.

In the description below, assume that a subject has been subject to a traumatic event, such as, for example, a blow to the head that a player in an athletic event might experience during a game (e.g., a football player, soccer player, lacrosse player, etc.), a driver of a car might experience during an accident, etc. Assume further that a user (e.g., a coach, a paramedic, a nurse, etc.), associated with blink reflex device 100, places blink reflex device 100 on the subject in a manner that enables blink reflex device 100 to obtain (e.g., detect, measure, record, etc.) a blink reflex response associated with the subject.

As shown in FIG. 10, process 1000 may include receiving a request to perform a test on a subject (block 1005) and detect an eye of the subject based on the request (block 1010). For example, blink reflex device 100 may receive an instruction to obtain a blink reflex response from the subject, such as when the user selects a particular button (e.g., to power up blink reflex device 100, etc.) on blink reflex device 100 and/or when blink reflex device 100 is placed on the subject, etc.). Blink reflex device 100 (e.g., sensor unit 215) may, based on receiving the instruction, may detect one or both eyes of the subject. For example, blink reflex device 100 may receive information associated with the eye of the subject (e.g., the face, one or both eyes, one or more eyelids, an area around an eye, etc. of the subject) and may determine whether the received information matches stored information (e.g., a visual signature of a standard eye stored in memory 410) associated with a particular eye, such as a video and/or image of a standard eye, eyelid, proximity thereof. In the event that the received information matches the stored information, blink reflex device 100 may use one or more known techniques to create a corneal reflection (e.g., corneal reflection 515 of FIG. 6A) on and/or within the eye to identify one or more tracking points associated with the subject (e.g., upper eyelid tracking point 525 (FIG. 6A), lower eye tracking point 530 (FIG. 6A), and/or some combination of upper and/or lower eyelid tracking points). Blink reflex device 100 may also, or alternatively, identify an initial location of the upper eyelid (e.g., based on upper eyelid tracking point 525) and/or the lower eyelid (e.g., based on lower eyelid tracking point 530) when the eye is in the open state. Blink reflex device 100 may output a notification that a tracking point has been identified. In the event that the received information does not match the stored information, blink reflex may output a notification that alerts the user that a tracking point cannot be identified.

As also shown in FIG. 10, process 1000 may include identifying a type of stimuli to apply to the subject (block 1015). For example, blink reflex device 100 may, based on identifying a tracking point associated with the eye, determine a type of stimuli that is to be used to obtain a blink reflex response from the subject. Blink reflex device 100 may, for example, receive an indication from the user that identifies the type of stimuli when the user selects a particular button on blink reflex device 100 (e.g., a button identifying mechanical, optical, acoustic, and/or electrical stimuli). Additionally, or alternatively, a particular type of stimuli, such as, for example, a mechanical stimuli (e.g., a puff of fluid, a pin prick, etc.) may be pre-programmed (e.g., as a default stimuli) into blink reflex device 100 by the user or during manufacturing. Blink reflex device 100 may also, or alternatively, receive an indication from the user (e.g., by selecting a specific button, preprogramming by user, preprogramming during manufacture, etc.) whether stimuli is to be provided to the right eye and/or proximity thereof, the left eye and/or proximity thereof, and/or both eyes and/or proximities thereof.

Additionally, or alternatively, the user may indicate whether a confounding operation is to be performed on the subject by selecting a certain button on blink reflex device 100. Blink reflex device 100 may include a default mode (e.g., preprogrammed by the user and/or during manufacturing) that does not include a confounding operation.

As further shown in FIG. 10, if the type of stimuli indicates a confounding operation (block 1020—YES), process 1000 may include performing a confounding operation on the subject (block 1025). For example, blink reflex device 100 may determine that a confounding operation is to be performed and may (e.g., using stimulator 102, confounder module 450, etc.) perform a confounding operation on the subject. The confounding operation may cause the subject respond to questions, audible sounds, a flash of light, etc. for the purpose of distracting the subject, which may preclude the subject from anticipating the stimuli and/or avoiding the surprise of the stimuli. Being surprised and/or startled by the stimuli may cause the subject to blink as a reflex in response to the stimuli rather than in anticipation of such stimuli, which may lead to inaccurate results. For example, blink reflex device 100 may perform the confounding operation by intermittently displaying one or more lights in the field of view of the subject and blink reflex device 100 and/or the user may direct the subject to identify when one of the lights is lit and/or the position of each light within the field of view. The confounding operation may cause the subject to focus concentration on one or more of the intermittent lights, which may preclude the subject from anticipating the stimuli. Additionally, or alternatively, blink reflex device 100 may also, or alternatively, perform the confounding operation using one or more sounds in which blink reflex device 100 and/or the user directs the subject to identify when a sound is made, which ear the sound is directed, whether the pitch is increasing or decreasing, etc. Blink reflex device 100 may also, or alternatively, perform other confounding operations (e.g., mechanical, electrical, etc.) by causing, for example, the subject to interact with a user interface displayed on user device 110 and/or blink reflex device 100 by answering questions, pointing to moving targets, etc.

As yet further shown in FIG. 10, if the type of stimuli does not indicate a confounding operation (block 1020—NO) or while performing the confounding operation on the subject (block 1025), process 1000 may include providing to the subject the stimuli based on the identified type of stimuli (block 1030). For example, blink reflex device 100 may determine that the identified type of stimuli indicates that a confounding operation is not to be performed on the subject. Blink reflex device 100 may, based on the determination that the confounding operation is not to be performed, provide the stimuli to the subject without performing the confounding operation. Alternatively, blink reflex device 100 may, while performing the confounding operation in a manner described above with respect to block 1025, provide stimuli to the subject while the confounding operation is being performed.

For example, blink reflex device 100 may provide a stimulus to the subject to cause the subject to reflexively blink in a manner that can be detected, monitored and/or recorded by blink reflex device 100. Additionally, or alternatively, blink reflex device 100 may stimulate the subject based on the identified type of stimuli. For example, if the type of stimuli corresponds to a mechanical stimulation, blink reflex device 100 (e.g., stimulator 102, mechanical module 410, etc.) may cause a puff of fluid (e.g., air, nitrogen, water, water vapor, etc.) to be directed and/or targeted to the selected eye of the subject (e.g., selected by the user and/or based on preprogramming). The puff of fluid may be associated with a particular volume, direction, pressure, velocity, acceleration, force, etc. that causes the subject to be startled or surprised. As also shown in FIG. 10, process 1000 may include obtaining first blink reflex information from the subject (block 1035) and obtaining second blink reflex information from the subject (block 1040). For example, blink reflex device 100 may, at a first time, track the manner in which the subject reflexively blinks as a result of providing the stimuli to the subject. The first time (e.g., T1) may correspond to a time during or after which the subject experiences a traumatic event associated with a blow or impact to the head. Blink reflex device 100 may track and/or record, as a function of time, the location along blink axis 520 (FIG. 6A) of one or more upper eyelid tracking points 525, lower eyelid tracking points 530 and/or some other tracking points relative to the initial location of such tracking points (e.g., when the eye is in the open state) to obtain information associated with the first blink reflex of the subject (sometimes referred to a “blink function”) in a manner similar to that described above with respect to FIGS. 5B and 8A-8D. Additionally, or alternatively, blink reflex device 100 may identify certain abnormal blink functions, such as a micro-blink and/or double-blink in a manner similar to that described above with respect to FIG. 9 and may discard, ignore, or erase a portion of the information associated with the first blink reflex to which the abnormal blink corresponds. Additionally, or alternatively, blink reflex device 100 may determine whether the subject potentially suffers from fatigue, cognitive impairment and/or impaired neurological function based on the information associated with the abnormal blink functions.

Additionally, or alternatively, blink reflex device 100 may obtain information associated with the blink of the subject when stimuli has not been provided to the subject, such as when the subject intentionally blinks (e.g., in response to a command from the user) and/or when the subject naturally blinks to lubricate the surface of the eye. Blink reflex device 100 may also, or alternatively, store the information, associated with the first blink reflex and/or first blink period, in a data structure (e.g., data structure 1100 of FIG. 11 to be described below) within a memory associated with blink reflex device 100 (e.g., memory 410) and/or may transmit the information, associated with first blink reflex and/or first blink period, to server 120 and/or database 130 for storage in a data structure.

Additionally, blink reflex device 100 may retrieve from a memory (e.g., memory 410), database 130 and/or server 120, information associated with a second blink reflex obtained at a prior, second point in time (e.g., T2). The information associated with the second blink reflex may have been obtained from the subject at the second time before the subject experienced the traumatic event and/or when the subject is known not to be suffering from a neurological condition. Additionally, or alternatively, the information, associated with the second blink reflex and/or second blink period, may correspond to a combination of one or more blink functions (e.g., an average, mean, median, etc.) of one or more other subjects (e.g., of the same or similar demographics, such as age, race, gender, etc. relative to the subject) at the second time when the other subjects are known not to be suffering from a neurological condition.

For example, as shown in FIG. 11, data structure 1100 may store information associated with the blink reflex of a subject and/or other subjects and may include a collection of fields, such as a subject info field 1105, a stimuli type field 1110, a confound field 1115, an eye identifier field 1120, a baseline time field 1100, a baseline blink reflex field 1130, a time field 1135, and a blink reflex field 1140. Additionally, or alternatively, data structure 1100 may be stored by blink reflex device 100 (e.g., memory 410), server 120, and/or database 130. The number of fields illustrated in FIG. 11, is provided for explanatory purposes only. In practice, there may be additional fields; fewer fields; different fields; or differently arranged fields than illustrated in FIG. 11.

Fields 1105 through 1130 may, for example, correspond to information previously obtained from the subject or other subjects prior to a traumatic event experienced by the subject. The other subjects may be associated with similar parameters or demographics as the subject (e.g., similar age, race, gender, size, weight, etc.). Fields 1135 and 114 may correspond to information obtained from the subject after the traumatic event is experienced by the subject. Subject info field 1105 may store information associated with a subject from which information associated with the first blink reflex and/or second blink reflex is obtained. For example, information, associated with the subject, may identify a name of the subject, an address of the subject, demographic information associated with the subject (e.g., age, gender, race, etc.), prior history (e.g., prior incidences of brain injury, neurological impairment, etc.), a unique identifier associated with the subject (e.g., a number, string, all or a portion of a social security number, etc.), etc. Subject info field 1105 may also, or alternatively, store information associated with one or more other subjects, known not to be suffering from a neurological condition, from which respective information, associated with a second blink reflex, is obtained. Additionally, or alternatively, the demographic information, associated with the other subjects, may be the same or similar to that of the subject.

Stimuli type field 1110 may store information that identifies a type of stimuli used to obtain the information associated with the first blink reflex or the second blink reflex. For example, the information that identifies the type of stimuli may identify if no stimuli was used (e.g., shown as S0 within stimuli type field 1110 of FIG. 11) or whether mechanical stimuli (e.g., shown as S1 within stimuli type field 1110 of FIG. 11), light stimuli (e.g., shown as S2 within stimuli type field 1110 of FIG. 11), acoustic stimuli (e.g., shown as S3 within stimuli type field 1110 of FIG. 11), and/or electrical stimuli (e.g., shown as S4 within stimuli type field 1110 of FIG. 11) was used to obtain the information associated with the first blink reflex and/or second blink reflex. Stimuli type field 1110 may also, or alternatively, store information that identifies whether the stimuli are provided to the left eye, right eye, both eyes, or proximity thereof of the subject.

Confound field 1115 may store information that identifies whether a confounding operation was performed on the subject to obtain the information associated with the first blink reflex or the second blink reflex (e.g., shown as C0 in field 1115 of FIG. 11 if a confounding operation was not performed, and C1 if a confounding operation was performed). Eye identifier field 1120 may store information that identifies whether the information associated with the first blink reflex or second blink reflex was obtained from the subject with respect to the left eye (e.g., shown as L within stimuli type field 1120 of FIG. 11), right eye (e.g., shown as R within stimuli type field 1120 of FIG. 11) or both eyes (e.g., shown as B within stimuli type field 1120 of FIG. 11). Baseline time field 1100 may store information (e.g., a date, time, etc.) that identifies a previous time (e.g., identified above as the second time and shown as T2 within baseline time field 1100 of FIG. 11) at which a blink reflex operation was performed (e.g., by blink reflex device 100) to obtain the information associated with the second blink reflex or second blink period of the subject or one or more other subject (e.g., other subjects associated with the same or similar demographics as the subject). The previous time may, for example, correspond to a time before the subject experienced a traumatic event and/or when it is known that the subject or the other subjects are known not to be suffering from a neurological condition. Baseline blink reflex field 1130 may store information associated with the second blink reflex and/or second blink period. The information associated with the second blink reflex and/or second blink period may, in a manner similar to that described above with respect to FIGS. 6B and 8A-8D, correspond to a blink function of the subject.

Time field 1135 may store information (e.g., a date, time, etc.) that identifies a time (e.g., identified above as the first time or a current time and shown as T1 in time field 1135 of FIG. 11) at which a blink reflex operation was performed (e.g., by blink reflex device 100) to obtain the information associated with the first blink reflex and/or first blink period of the subject. The time may, for example, correspond to a particular time during or after which the subject experiences a traumatic event and/or when it is known that the subject is suffering from a neurological condition. Blink reflex field 1140 may store information associated with the first blink reflex and/or the first blink period of the subject. The information associated with the first blink reflex and/or first blink period may, in a manner similar to that described above with respect to FIGS. 6B and 8A-8D, correspond to a blink function of the subject that identifies a vertical distance that one or more eyelids, of the subject, move during one or more blinks by the subject as a function of time during which the one or more blinks are measured.

By way of an example associated with dashed ellipse 1152 of FIG. 11, at the second time (e.g., T2), blink reflex device 100 may have previously obtained information associated with the second blink reflex and/or second blink response of the subject without stimuli to the subject (e.g., S0), without performing a confounding operation (e.g., NC), and/or from both eyes of the subject (e.g., B) and may store such information in data structure 1100 (e.g., shown as BTB0).

Additionally, or alternatively, at the first time (e.g., T1) that occurs after the second time (e.g., T2) and after the subject has experienced a traumatic event or is known to suffer from a degenerative neurological condition, blink reflex device 100 may obtain information associated with the first blink reflex and/or first blink period of the subject under the same conditions as described in the previous paragraph. Blink reflex device 100 may store such information in data structure 1100 (e.g., shown as BT0).

Additionally, or alternatively, as shown with respect to dashed ellipse 1154 of FIG. 11, information associated with the second blink reflex and/or second blink period of the subject, may have been previously obtained at the second time (e.g., T2) under the same conditions as those described above, except in this case the subject was being confounded by blink reflex device 100 (e.g., shown as C in confound field 1115). Blink reflex device 100 may, in this example, store the information associated with second blink reflex in data structure 1100 (e.g., shown as BTB1). Blink reflex device 100 may, during the first time (e.g., T1), obtain information associated with the first blink reflex and/or the first blink period under the confounding conditions described in this example, and may store such information in data structure 1100 (e.g., shown as BT1).

Additionally, or alternatively, as shown with respect to dashed ellipse 1156 of FIG. 11, at the second time (e.g., T2), blink reflex device 100 may have previously obtained information associated with the second blink reflex and/or second blink period in one or more separate measurements of the right eye (e.g., shown as R) and of the left eye of the subject (e.g., shown as L), by providing a first stimuli to the subject (e.g., a mechanical stimuli shown as S1), and performing a confounding operation on the subject (e.g., shown as C). Blink reflex device 100 and may store such information in data structure 1100 (e.g., shown as RTB1 for the right eye and LBT1 for the left eye). Additionally, or alternatively, at the first time (e.g., T1) that occurs after the second time and after the subject has experienced a traumatic event or is known to suffer from a degenerative neurological condition, blink reflex device 100 may obtain information associated with the first blink reflex and/or first blink period of the subject (e.g., for the right eye and separately for the left eye) under the same conditions as described immediately above and may store such information in data structure 1100 (e.g., shown as RT1 for the right eye and LT1 for the left eye).

Additionally, or alternatively, as shown with respect to dashed ellipse 1158, blink reflex device 100 may, at the second time (e.g., T2), have previously obtained information associated with second blink reflex and/or second blink period from the right and/or left eye based on the conditions set forth in the previous example with respect to dashed ellipse 1156, except that no confounding operation is performed (e.g., NC). Blink reflex device 100 may store such information in data structure 1100 (e.g., shown as RTB2 for the right eye and LTB2 for the left eye). Additionally, or alternatively, blink reflex device 100 may, at the first time (e.g., T1), obtain information associated with the first blink reflex and/or first blink period of the subject under the same conditions as described immediately above and may store such information in data structure 1100 (e.g., shown as RT2 for the right eye and LT2 for the left eye). Blink reflex may also, or alternatively, have previously obtained (e.g., at T1) and/or may obtain (e.g., at T2) other information associated with the first blink reflex/first blink period or the second blink reflex/second blink period based on other types of stimuli (e.g., shown as S2, S3, S4, etc.) and may store such information in data structure 1100 (e.g., as shown by dashed rectangle 1160 of FIG. 1100).

Returning to FIG. 10, process 1000 may include determining a change in the blink reflex based on the first blink reflex information and the second blink reflex information (block 1045). For example, blink reflex device 100 (e.g., processing unit 400) may compare the information associated with the first blink reflex and/or first blink period of the subject with the information associated with the second blink reflex and/or second blink period. The information may be associated with the second blink reflex or second blink period may have been obtained from the subject and/or one or more other subjects. In the latter case, the information associated with the second blink reflex and/or second blink period may be based on a combination of information taken from one or more second blink reflexes and/or second blink periods of one or more other subjects (e.g., based on an average, mean, median, etc.), obtained under the same and/or similar conditions (e.g., type of stimuli, with or without confounding, etc.). Blink reflex device 100 may identify an amount of difference or change between the information associated with the first blink reflex and/or blink period and the information associated with the second blink reflex and/or blink period. For example, blink reflex device 100 may, with respect to conditions in which the subject is not stimulated or confounded, compare the information associated with the first blink reflex or blink period of the subject (e.g., BT0 in the case of both eyes being measured) with the information associated with the second blink reflex and/or blink period (e.g., BTB0), to identify an amount of change or difference in the blink reflex and/or blink period under such conditions (e.g., ΔB0=|BT0−BTB0|). Additionally, or alternatively, blink reflex device 100 may, with respect to conditions in which the subject is not stimulated but is confounded, compare the information associated with the first blink reflex and/or first blink period of the subject (e.g., BT1) with the information associated with the second blink reflex and/or second blink period (e.g., BTB1), to identify an amount of change in the blink reflex and/or blink period (e.g., ΔB1) under such conditions (e.g., ΔB1=|BT1−BTB1|).

Additionally, or alternatively, blink reflex device 100 may, with respect to conditions in which the subject is being stimulated (e.g., using mechanical stimulation) and is being confounded, compare the information associated with the first blink reflex and/or blink period of the subject (e.g., RT1 in the case of the right eye) with the information associated with the second blink reflex and/or blink period (e.g., RTB1), to identify an amount of change in the blink reflex and/or blink period of the right eye under such conditions (e.g., ΔR1=|RT1−RTB1|). Additionally, or alternatively, blink reflex device 100 may, with respect to conditions in which the subject is stimulated (e.g., using mechanical stimulation) but is not confounded, compare the information associated with the first blink reflex and/or blink period of the subject (e.g., RT2 in the case of the right eye) with the information associated with the second blink reflex and/or blink period (e.g., RTB2), to identify an amount of change in the blink reflex and/or blink period of the right eye under such conditions (e.g., ΔR2=|RT2−RTB2|).

Blink reflex device 100 may perform a similar comparison for the right eye, left eye and/or both eyes for other conditions associated with different types of stimuli (e.g., light, acoustic, electrical, etc.) with and/or without confounding the subject and may determine the amount of change or difference in the blink reflex and/or blink period of the subject.

Additionally, or alternatively, blink reflex device 100 may, under certain conditions, compare information associated with the first blink reflex or blink period for the right eye with information associated with the first blink reflex and/or blink period for the left eye to identify any asymmetry in such first blink reflexes. For example, blink reflex device 100 may, with respect to conditions in which the subject is stimulated (e.g., using mechanical stimulation) and is confounded, compare the information associated with the first blink reflex and/or blink period for the right eye (e.g., RT1) with the information associated with the first blink reflex and/or blink period for the left eye (e.g., LT1) to identify an amount of difference in the first blink reflex and/or blink period of the right eye relative to that of the left eye (e.g., ΔLR1) under such conditions (e.g., ΔLR1=|RT1−LT1|). Blink reflex device 100 may perform a similar comparison for other conditions associated with different types of stimuli (e.g., light, acoustic, electrical, etc.) with or without confounding the subject and may determine the amounts of difference in the first blink reflex between the ipsilateral eye and contralateral eyes of the subject. Additionally, or alternatively, blink reflex device 100 may store one or more values, associated with the change in blink reflex and/or blink period in first blink reflex in data structure 1100 of FIG. 11.

As shown in FIG. 10, if the amount of change or difference in the blink reflex is less than a first threshold, and not greater than or equal to a second threshold (block 1050—YES<FIRST THRESHOLD), process 1000 may include outputting an indication that brain injury is unlikely (block 1055). For example, blink reflex device 100 may determine whether the amount of change in the blink reflex and/or blink period, of the subject, before and after the subject experiences trauma (e.g., a blow to the head, etc.) is less than a first threshold. In the event that the amount of change is less than the first threshold, blink reflex device 100 may output an indication that it is unlikely that the subject suffers from a neurological condition. Such an indication may enable the user, of blink reflex device 100, to decide to allow the subject to resume normal activity, such as, for example, return to the playing field, operate an automobile, return to work, etc.

For example, blink device 100 may retrieve, from a data structure (e.g., data structure 1200 of FIG. 12) within a memory associated with blink reflex device 100, server 120, and/or database 130, information identifies one or more thresholds, associated with conditions under which information associated with a blink reflex is obtained from a subject. The thresholds may be used by blink reflex device 100 to determine if the subject suffers from a neurological condition and/or the severity thereof. As shown in FIG. 12, data structure 1200 may include a collection of fields such as a no impairment field 1210, a some impairment field 1215, and a significant impairment field 1220. The number of fields illustrated in FIG. 12, is provided for explanatory purposes only. In practice, there may be additional fields; fewer fields; different fields; or differently arranged fields than illustrated in FIG. 12.

No impairment field 1210 may store information that identifies a first threshold (e.g., shown as br1, nbr1, c1, nc1, c1r1, cn1r1, etc. in FIG. 12) that corresponds to a time period, associated with a change in blink reflex and/or blink period, below which would indicate that the subject does not suffer from a neurological condition. For example, if the change in blink reflex of the subject is less than a first threshold for the conditions measured by blink reflex device 100, blink reflex device 100 may determine that it is not likely that the subject is suffering from a brain injury or degenerative neurological impairment.

Some impairment field 1215 may store information that identifies a range of time, from a first threshold to a second threshold (e.g., shown as br2, nbr2, c1, nc2, c1r2, cn1r2, etc. in FIG. 12), associated with a change in blink reflex and/or blink period, within which would indicate that the subject is suffering from a neurological condition. The second threshold may be greater than the first threshold. For example, if the change in blink reflex of the subject is not less than a first threshold and is less than a second threshold for the conditions measured by blink reflex device 100, blink reflex device 100 may determine that it is likely that the subject is suffering from a brain injury or degenerative neurological impairment.

Significant impairment field 1220 may store information that identifies the second threshold that corresponds to a time period, associated with a change in blink reflex and/or blink period, above which would indicate that the subject is suffering from a significant brain injury or degenerative neurological condition. For example, if the change in blink reflex of the subject is not less than a second threshold, blink reflex device 100 may determine that it is likely that the subject is suffering from a significant neurological condition.

Returning to FIG. 10 and by way of example, with respect to conditions in which the subject is not stimulated or confounded, blink reflex device 100 may determine whether the amount of change in blink reflex and/or blink period (e.g., ΔB0) is less than a first threshold (e.g., br1) associated with such conditions (e.g., shown as ΔB0<br1 in no impairment field 1210 of FIG. 12), where br1 represents the first threshold for conditions in which the subject is not stimulated or confounded). In the event that the amount of change is less than the first threshold, blink reflex device 100 may output an indication that it is unlikely that the subject suffers from a neurological condition. Additionally, or alternatively, with respect to conditions in which the subject is not stimulated but is confounded, blink reflex device 100 may determine whether the amount of change in blink reflex and/or blink period (e.g., ΔB1) is less than a first threshold associated with such conditions (e.g., shown as ΔB1<nbr1 in no impairment field 1210 of FIG. 12, where nbr1 represents the first threshold for conditions in which the subject is not stimulated but is confounded).

Additionally, or alternatively, with respect to conditions in which the subject is stimulated and confounded, blink reflex device 100 may determine whether the amount of change in blink reflex and/or blink period (e.g., ΔR1 for the right eye or ΔL1 for the left eye) is less than a first threshold associated with such conditions (e.g., shown as ΔR1<c1 or ΔL1<c1 in no impairment field 1210 of FIG. 12, where c1 represents the first threshold for conditions in which the subject is stimulated and confounded). The change in blink reflex and/or blink period for conditions associated with other types of stimuli (e.g., light, acoustic, electrical, etc.) and confounding may be compared, in the manner described above, to other first thresholds for such conditions associated with the other types of stimuli and confounding.

Additionally, or alternatively, with respect to conditions in which the subject is stimulated but not confounded, blink reflex device 100 may determine whether the amount of change in blink reflex and/or blink period (e.g., ΔR2 for the right eye or ΔL2 for the left eye) is less than a first threshold associated with such conditions (e.g., shown as ΔR2<nc1 or ΔL2<nc1 in no impairment field 1210 of FIG. 12, where nc1 represents the first threshold for conditions in which the subject is stimulated but not confounded). The change in blink reflex and/or blink period for conditions associated with other types of stimuli (e.g., light, acoustic, electrical, etc.) but no confounding may be compared, in the manner described above, to other first thresholds for such conditions associated with the other types of stimuli and no confounding.

Additionally, or alternatively, with respect to conditions in which the subject is stimulated and confounded, blink reflex device 100 may determine whether the amount of difference between the first blink reflex and/or blink period of the ipsilateral and contralateral eye (e.g., ΔLR1) is less than a first threshold associated with such conditions (e.g., shown as ΔLR1<c1r1 in no impairment field 1210 of FIG. 12, where c1r1 may represent the first threshold for conditions in which the subject is stimulated and confounded). The change in the first blink reflex between the ipsilateral and contralateral eye for conditions associated with other types of stimuli (e.g., light, acoustic, electrical, etc.) and confounding may be compared, in the manner described above, to other first thresholds for such conditions associated with the other types of stimuli and confounding. Additionally, or alternatively, with respect to conditions in which the subject is stimulated but not confounded, blink reflex device 100 may determine whether the amount of difference between the first blink reflex and/or blink period of the ipsilateral and contralateral eye (e.g., ΔLR2) is less than a first threshold associated with such conditions (e.g., shown as ΔLR2<nc1r1 in no impairment field 1210 of FIG. 12, where nc1r1 may represent the first threshold for conditions in which the subject is stimulated but not confounded).

The change in the first blink reflex between the ipsilateral and contralateral eye for conditions associated with other types of stimuli (e.g., light, acoustic, electrical, etc.) and confounding may be compared, in the manner described above, to other first thresholds for such conditions associated with the other types of stimuli and confounding. In the event that each of the amounts of change in blink reflex and/or blink period are less than the respective first thresholds as described above, blink reflex device 100 may output an indication that it is unlikely that the subject suffers from a neurological condition. Additionally, or alternatively, if the difference in first blink reflex, between the ipsilateral and contralateral eye, is less than the corresponding first threshold, blink reflex device 100 may output an indication that it is unlikely that the subject suffers from a neurological condition.

As also shown in FIG. 10, if the change in the blink reflex is not less than the first threshold or not greater than or equal to the second threshold (block 1050—NO), process 1000 may include outputting an indication that brain injury is likely (block 1060). For example, blink reflex device 100 may determine whether the amount of change in the blink reflex and/or blink period, of the subject, before and after the subject experiences trauma indicates that the subject has suffered a brain injury and/or a degenerative neurological condition. Based on a determination that the subject suffers from a brain injury and/or a degenerative neurological condition, blink reflex device 100 may output an indication that it is likely that the subject suffers from a neurological condition. Such an indication may enable the user, of blink reflex device 100, to decide to prohibit the subject from resuming normal activity, such as, for example, prohibiting a subject from returning to the playing field, operating an automobile, returning to work, etc.

For example, with respect to conditions in which the subject is not stimulated or confounded, blink reflex device 100 may determine whether the amount of change in blink reflex and/or blink period (e.g., ΔB0) is not less than the first threshold (e.g., br1) associated with such conditions and is not greater than or equal to a second threshold associated with such conditions (e.g., shown as br1≤ΔB0<br2 in some impairment field 1215 of FIG. 12, where br2 represents the second threshold for conditions in which the subject is not stimulated or confounded). In the event that the amount of change is not less than the first threshold and is not greater than or equal to the second threshold, blink reflex device 100 may output an indication that it is likely that the subject suffers from a neurological condition. Additionally, or alternatively, with respect to conditions in which the subject is not stimulated but is confounded, blink reflex device 100 may determine whether the amount of change in blink reflex and/or blink period (e.g., ΔB1) is not less than a first threshold associated with such conditions and is not greater than or equal to a second threshold associated with such conditions (e.g., shown as nbr1≤ΔAB1<nbr2 in some impairment field 1215 of FIG. 12, where nbr2 represents the second threshold for conditions in which the subject is not stimulated but is confounded).

Additionally, or alternatively, with respect to conditions in which the subject is stimulated and confounded, blink reflex device 100 may determine whether the amount of change in blink reflex and/or blink period (e.g., ΔR1 for the right eye or ΔL1 for the left eye) is not less than a first threshold associated with such conditions and is not greater than or equal to a second threshold associated with such conditions (e.g., shown as c1≤ΔR1<c2 in some impairment field 1215 of FIG. 12, where c2 represents the second threshold for conditions in which the subject is stimulated and confounded) (e.g., shown as c1≤ΔL1<c2 in some impairment field 1215 of FIG. 12). The change in blink reflex for conditions associated with other types of stimuli (e.g., light, acoustic, electrical, etc.) and confounding may be compared, in the manner described above, to other first thresholds and second thresholds for such conditions associated with the other types of stimuli and confounding.

Additionally, or alternatively, with respect to conditions in which the subject is stimulated but not confounded, blink reflex device 100 may determine whether the amount of change in blink reflex and/or blink period (e.g., ΔR2 for the right eye or ΔL2 for the left eye) is not less than a first threshold associated with such conditions and is not greater than or equal to a second threshold associated with such conditions (e.g., shown as nc1≤ΔR2<nc2 in some impairment field 1215 of FIG. 12, where nc2 represents the second threshold for conditions in which the subject is stimulated but not confounded) (e.g., shown as nc1≤ΔL2<nc2 in some impairment field 1215 of FIG. 12). The change in blink reflex for conditions associated with other types of stimuli (e.g., light, acoustic, electrical, etc.) but no confounding may be compared, in the manner described above, to other first thresholds and second thresholds for such conditions associated with the other types of stimuli and confounding.

Additionally, or alternatively, with respect to conditions in which the subject is stimulated and confounded, blink reflex device 100 may determine whether the amount of difference between the first blink reflex and/or blink period of the ipsilateral and contralateral eye (e.g., ΔLR1) is not less than a first threshold associated with such conditions and not greater than or equal to a second threshold associated with such conditions (e.g., shown as c1r1≤ΔLR1<c1r2 in some impairment field 1215 of FIG. 12, where c1r2 represents the second threshold for conditions in which the subject is stimulated and confounded). The change in the first blink reflex between the ipsilateral and contralateral eye for conditions associated with other types of stimuli (e.g., light, acoustic, electrical, etc.) and confounding may be compared, in the manner described above, to other first thresholds and/or second thresholds for such conditions associated with the other types of stimuli and confounding. Additionally, or alternatively, with respect to conditions in which the subject is stimulated but not confounded, blink reflex device 100 may determine whether the amount of difference between the first blink reflex and/or blink period of the ipsilateral and contralateral eye (e.g., ΔLR2) is not less than a first threshold associated with such conditions and is not greater than or equal to a second threshold associated with such conditions (e.g., shown as nc1r1≤ΔLR2<nc1r2 in some impairment field 1215 of FIG. 12, where nc1r2 represents the second threshold for conditions in which the subject is stimulated but not confounded). The change in the first blink reflex and/or blink period between the ipsilateral and contralateral eye for conditions associated with other types of stimuli (e.g., light, acoustic, electrical, etc.) and confounding may be compared, in the manner described above, to other first thresholds for such conditions associated with the other types of stimuli and confounding.

In the event that each of the amounts of change in blink reflex are not less than the respective first thresholds and are not greater than or equal to the respective second thresholds as described above, blink reflex device 100 may output an indication that it is likely that the subject suffers from a neurological condition. Additionally, or alternatively, if the difference in first blink reflex, between the ipsilateral and contralateral eye, is not less than the corresponding first threshold and is not greater than or equal to the corresponding second threshold, blink reflex device 100 may output an indication that it is likely that the subject suffers from a neurological condition.

As further shown in FIG. 10, if the change in the blink reflex is not less than the first threshold and is greater than or equal to the second threshold (block 1050—YES≥SECOND THRESHOLD), process 1000 may include outputting an indication that significant brain injury is likely (block 1065). For example, blink reflex device 100 may determine whether the amount of change in the blink reflex and/or blink period of the subject, before and after the subject experiences trauma, indicates that the subject is suffering from a significant neurological condition. Based on a determination that the subject suffers from a significant neurological condition, blink reflex device 100 may output an indication that it is likely that the subject suffers from a significant neurological condition. Such an indication may enable the user, of blink reflex device 100, to decide to prohibit the subject from resuming normal activity and/or by seeking immediate medical treatment for the subject.

For example, with respect to conditions in which the subject is not stimulated or confounded, blink reflex device 100 may determine whether the amount of change in blink reflex and/or blink period (e.g., ΔB0) is greater than or equal to a second threshold (e.g., br2) associated with such conditions (e.g., shown as br2≤ΔB0 in significant impairment field 1220 of FIG. 12). In the event that the amount of change is greater than or equal to the second threshold, blink reflex device 100 may output an indication that it is likely that the subject suffers from a significant neurological condition. Additionally, or alternatively, with respect to conditions in which the subject is not stimulated but is confounded, blink reflex device 100 may determine whether the amount of change in blink reflex and/or blink period (e.g., ΔB1) is greater than or equal to a second threshold associated with such conditions (e.g., shown as nbr2≤ΔB1 in significant impairment field 1220 of FIG. 12).

Additionally, or alternatively, with respect to conditions in which the subject is stimulated and confounded, blink reflex device 100 may determine whether the amount of change in blink reflex and/or blink period (e.g., ΔR1 for the right eye or ΔL1 for the left eye) is greater than or equal to a second threshold associated with such conditions (e.g., shown as c2≤ΔR1 or c2≤ΔL1 in significant impairment field 1220 of FIG. 12). The change in blink reflex and/or blink period for conditions associated with other types of stimuli (e.g., light, acoustic, electrical, etc.) and confounding may be compared, in the manner described above, to other second thresholds for such conditions associated with the other types of stimuli and confounding.

Additionally, or alternatively, with respect to conditions in which the subject is stimulated but not confounded, blink reflex device 100 may determine whether the amount of change in blink reflex and/or blink period (e.g., ΔR2 for the right eye or ΔL2 for the left eye) is greater than or equal to a second threshold associated with such conditions (e.g., shown as nc2≤ΔR2 or nc2≤ΔL2 in no impairment field 1210 of FIG. 12). The change in blink reflex and/or blink period for conditions associated with other types of stimuli (e.g., light, acoustic, electrical, etc.) but no confounding may be compared, in the manner described above, to other second thresholds for such conditions associated with the other types of stimuli and no confounding.

Additionally, or alternatively, with respect to conditions in which the subject is stimulated and confounded, blink reflex device 100 may determine whether the amount of difference between the first blink reflex and/or blink period of the ipsilateral eye and contralateral eye (e.g., ΔLR1) is greater than or equal to a second threshold associated with such conditions (e.g., shown as c1r1≤ΔLR1 in significant impairment field 1220 of FIG. 12). The change in the first blink reflex and/or blink period between the ipsilateral eye and contralateral eye for conditions associated with other types of stimuli (e.g., light, acoustic, electrical, etc.) and confounding may be compared, in the manner described above, to other second thresholds for such conditions associated with the other types of stimuli and confounding. Additionally, or alternatively, with respect to conditions in which the subject is stimulated but not confounded, blink reflex device 100 may determine whether the amount of difference between the first blink reflex and/or blink period of the ipsilateral eye and contralateral eye (e.g., ΔLR2) is greater than or equal to a second threshold associated with such conditions (e.g., shown as nc1r1≤ΔLR2 in significant impairment field 1220 of FIG. 12). The change in the first blink reflex and/or blink period between the ipsilateral eye and contralateral eye for conditions associated with other types of stimuli (e.g., light, acoustic, electrical, etc.) and confounding may be compared, in the manner described above, to other second thresholds for such conditions associated with the other types of stimuli and confounding.

In the event that each of the amounts of change in blink reflex and/or blink period are greater than or equal to the respective second thresholds as described above, blink reflex device 100 may output an indication that it is likely that the subject suffers from a significant neurological condition. Additionally, or alternatively, if the difference in first blink reflex, between the ipsilateral eye and contralateral eye, is greater than or equal to the corresponding second threshold, blink reflex device 100 may output an indication that it is likely that the subject suffers from a significant neurological condition.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

The following study examined the utility of non-invasive measurements of the blink reflex as a diagnostic test for concussion. The blink reflex is a primitive brainstem response to an external stimulus, such as air, visual cues or electrical signals, which is affected by multiple neurological disorders, including those that affect the dopaminergic circuit that controls the eyelid. Previous studies using electromyography have shown that diffuse axonal injury and exercise result in measurable changes in the blink reflex. High speed videography was employed with air puffs to determine whether a head impact suspected of causing a concussion results in changes in the blink reflex that can be detected non-invasively. Further, the study assessed whether changes in the blink reflex could discriminate between players receiving a head impact and those who had simply been involved in physical activity.

### Methods

Twenty-six division I athletes between the ages of 18-22 were included in this study (24 Male, 2 Female; 24 football players; 1 soccer player; 1 volleyball player). Prior to the beginning of the study, subjects read a document which described the procedures of this study. Pre-season baseline data including, an athletic history and physical examination, were collected on each subject. Baseline Biodex Balance System SD (Biodex Medical Systems, Inc., Shirley, N.Y.) assessments and baseline symbols modalities tests were also completed on each athlete. Along with these routine pre-season assessments, the study utilized an embodiment of the invention called the Blink Reflexometer (described below) to obtain baseline blink reflex data on each subject. Data was collected throughout the season if a concussion occurred or was suspected to occur. The specific measurements collected after a concussion included: blink reflex, the Acute Concussion Evaluation (ACE), and a symptoms and severity checklist to assess symptoms such as headache, nausea, difficulty remembering, heart rate, and blood pressure.

The Blink Reflexometer includes a high-speed videography-based device used to trigger, record and analyze a blink reflex. The Blink Reflexometer consists of a mask, a stimulation system, a housing unit, a camera, an external controller and processor, and a user interface (FIG. 13). To capture the blink reflex response, a subject placed their face against the mask and aligned their eyes to internal mirrors within the housing unit. The test administrator, who was able to view real-time images of the subject's eyes on the user interface, confirmed proper eye alignment prior to manually commencing the video-recorded test. The video segment was captured at 280 FPS (frames per second) which gave each frame a 3.5 ms window. To illicit the startle response, the stimulation system administered, via adjustable nozzles, one to three air puffs over a 20 second time frame to the outer corner of either the right or left eye, with the laterality and timing of the puffs randomly assigned. Microphones (CME-1538-100LB, CUI Inc., Tualatin, Oreg.), connected to the processor, were positioned at the exit of the nozzles to capture the sound of the air exiting. The microphone recording provided a time stamp of stimulation delivery to the eye, which was used for blink reflex parameter calculations. After the 20-second test was administered, the subject had time to rest (approximately 20 seconds) while the software processed the video. Each subject received a total of two or three 20 second tests during a session, with results of the sessions analyzed and means recorded.

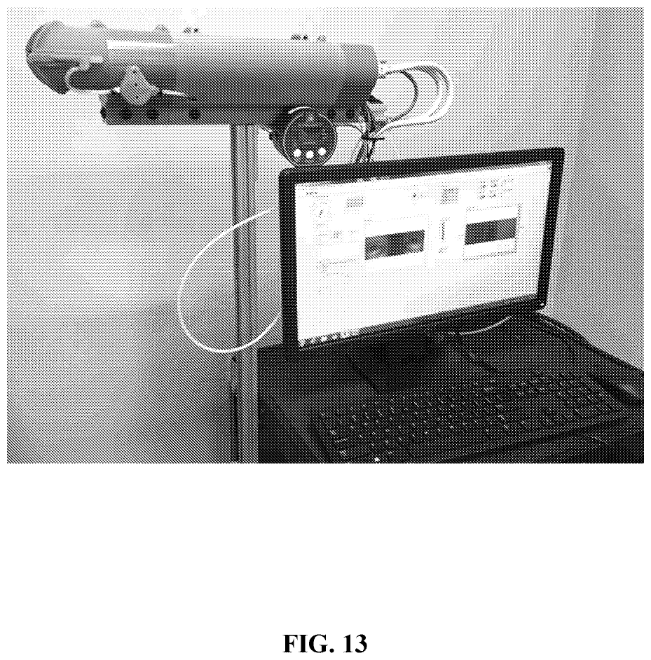

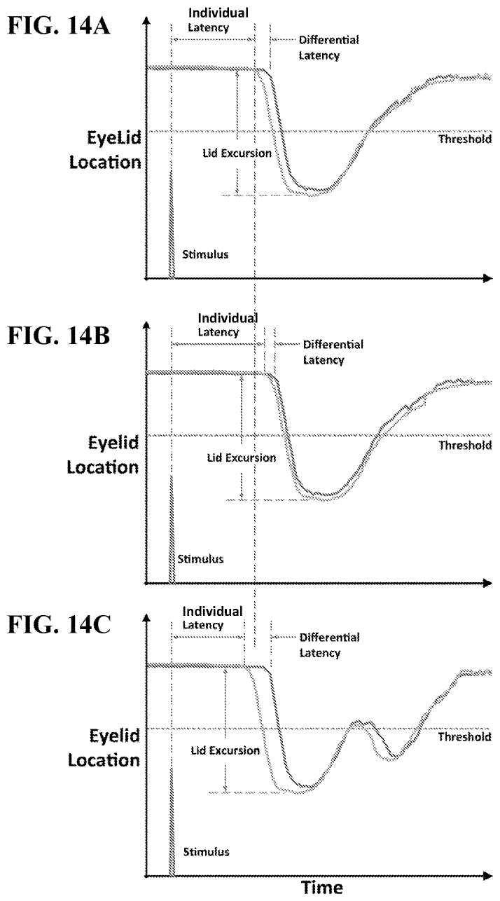

Processing of the video included detecting the edge of the both eyelids using custom LabVIEW software (National Instruments, Austin, Tex.). The program then tracked, using an edge detection function, the vertical positions of each eyelid through the entire image sequence. Frames were converted to time based on the collection frequency. For each eyelid, pixel location per time was used to chart a displacement profile (FIG. 14A). To establish reference positions for blink parameter measurements, a rest position and threshold were defined as follows:

Tonic Lid Position: moving average of the pixel location of the top eyelid when not in a blink

Threshold: 20 pixels below tonic lid position

From the displacement profile for each eyelid, differences within and between subjects were assessed for the following parameters:

Ipsilateral: stimulated side

Contralateral: side opposite the stimulation

Individual Latency: time differential between stimulation and ipsilateral eye movement

Differential Latency: time differential between the start of ipsilateral eye movement and the start of contralateral eye movement

Lid Excursion: distance traveled by the eyelid from the tonic lid position to closed position measured in pixels

Lid Velocity: average eyelid speed (pixels/sec) in first 7 frames following start of eyelid movement

Time to Close: time for lid to travel from tonic lid position to the closed position

Time to Open: time for lid to travel from closed position back to tonic lid position

Total Blink Time: time from start of eyelid movement until it returns to its tonic lid position

Time under Threshold: time that the eyelid spends below the threshold position

Number of Oscillations: cycles of up and down upper eyelid movement after a stimulated blink

Delta 30: time difference between the ipsilateral eye and contralateral eye after the lids had moved 30 pixels from the tonic lid position.

Subjects were divided into two groups, “Head Impact” and “Control”, during the study depending on if they were suspected of having suffered a concussive event during the study period. Pre-season blink reflex measurements were taken to establish “baseline” parameters for each subject. Control subjects were also tested after a practice to collect “active” blink reflex parameters. Head Impact subjects were tested as soon after the head impact as possible (1-48 hours) to collect “Post-Head Impact” blink reflex parameters.

### Statistical Analysis

Athlete measurements were defined into one of 4 categories: (1) Baseline Control, (2) Active Control, (3) Baseline Head Impact, and (4) Post-Head Impact. A linear mixed model (LMM) was used to account for the correlation within subjects which resulted from repeated measures captured on the same subject using a random subject effect. The LMMs included a main effect for athlete type and a random subject effect to account for correlation between measures collected on the same subject. Different correlation structures (e.g. compound symmetry, unstructured) and the final correlation structure was selected based on Akaike's Information Criterion. Comparisons between baseline and active measures within Control athletes, baseline and post-head impact measures within Head Impact athletes, and between the differences in the changes observed in Control and Head Impact athletes were assessed using a series of linear contrast statements from the models. All model assumptions were checked graphically and log transformations were considered if model assumptions were violated. Blink measures that met the statistical assumptions for an LMM model included individual latency, differential latency, delta 30, lid excursion, and lid velocity. The blink measures time to open, time to close, time under threshold, number of oscillations, time to first oscillation, and total blink time were all log transformed in the analysis to meet statistical assumptions. All analyses were conducted in SAS 9.4 (SAS Institute, Inc., Cary, N.C.).

### Results

Data were collected on 16 athletes with at least one head impact suspected of resulting in a concussion (2 players had 2 head impacts; 1 player had 3 impacts) and 10 control players who were age matched and had no history of concussive events.

(1) Changes in Blink Parameters Resulting from Physical Activity in Control Athletes

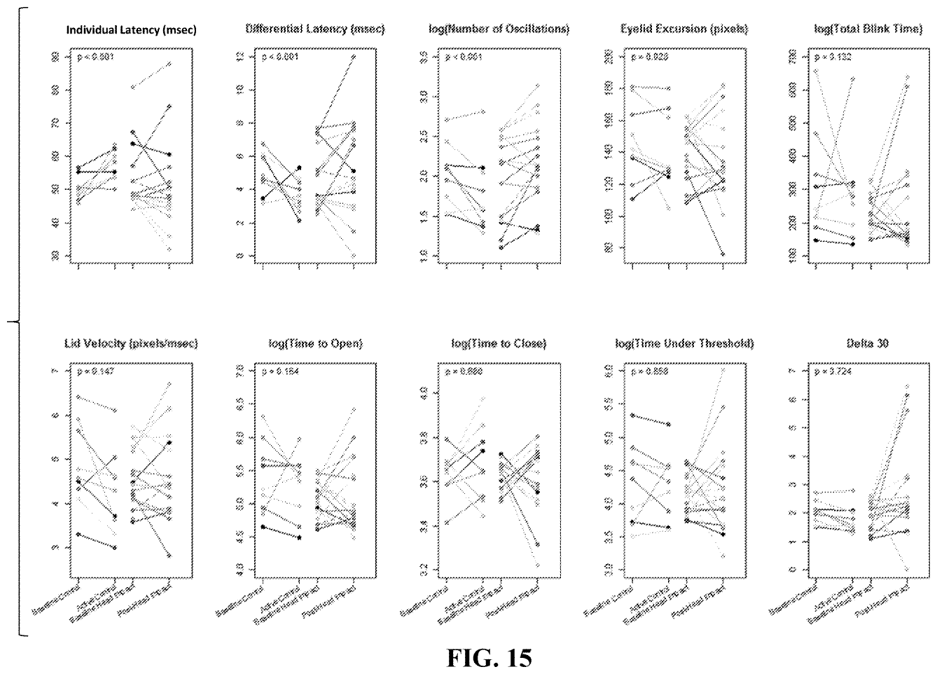

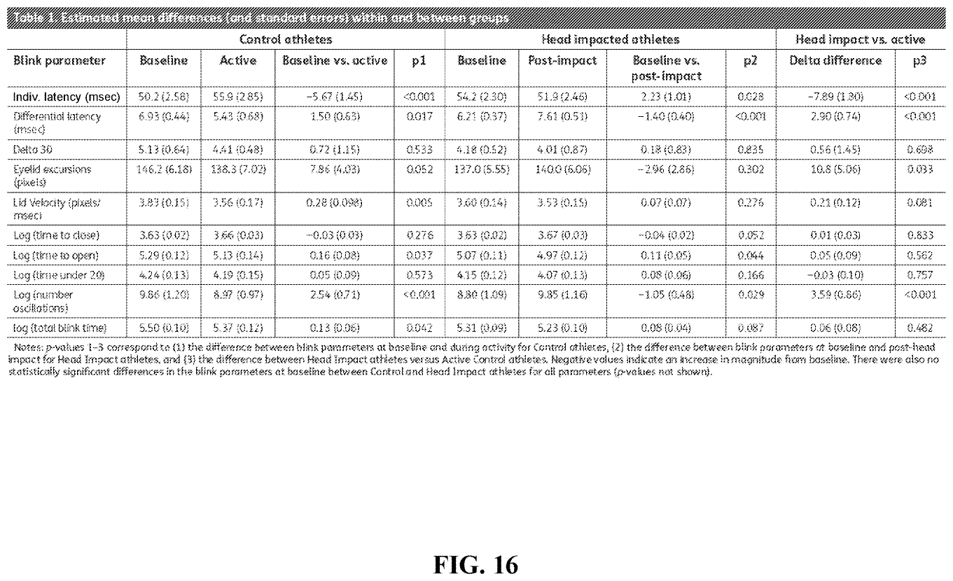

Significant differences in blink parameters between baseline and active measurements in Control athletes were observed for individual latency, differential latency, lid velocity, log of time to open, log of the number of oscillations, and log of total blink time (FIGS. 15 and 16). Specifically, active play resulted in increased in individual latency (p<0.001), decreased differential latency (p=0.017), decreased lid velocity (p=0.005), longer time to open (p=0.037), fewer oscillations (p=0.002), and longer total blink duration (p=0.042) (FIG. 14B). There were no significant differences in delta 30, lid excursion, log of time to close, or log of time under threshold.

(2) Changes in Blink Parameters Resulting from a Head Impact

Significant differences between blink parameters measured at baseline and post-head impact in Head Impact athletes were observed for individual latency, differential latency, lid velocity, log of time to close, and log of number of oscillations (FIGS. 15 and 16). Specifically, head impacts resulted in decreased individual latency (p=0.017), increased differential latency (p=0.001), decreased log of the time to close (p=0.012); and increased oscillations (p=0.008) (FIG. 14C). There were no significant differences in delta 30, lid excursion, log of time to open, log of time under threshold, or log of total blink time.

(3) Discrimination between Active Controls and Post-Head Impact Athletes

Significant differences between the changes observed in blink parameters in Control and Head Impact athletes were observed for individual latency, differential latency, lid excursion, log of time to open, log of the number of oscillations, and log of total blink time.

Head Impact athletes had decreased individual latency post-head impact compared to baseline, while Active Control athletes had increased individual latency after activity relative to baseline (p<0.001). Head Impact athletes had increased differential latency after post-head impact relative to baseline, while Active Control athletes had decreased differential latencies after activity relative to baseline (p<0.001). Head Impact athletes had larger lid excursions post-head impact relative to baseline, while Active Control athletes had smaller lid excursions after activity relative to baseline (p=0.028). Head Impact athletes had increased number of oscillations post-head impact, while Active Control athletes exhibited a decrease in the number of oscillations relative to baseline (p<0.001).

### Discussion

This study utilized a novel device to assess whether head impacts suspected of resulting in a concussion produced changes in the blink reflex that could be detected using non-invasive measurements, and whether those changes could discriminate between a concussive event and mere physical activity, such that the approach has potential as a field-side diagnostic tool. The results show that athletes who experienced a head impact had a decrease in individual latency, increase in differential latency, larger lid excursions, and an increase in oscillations post injury as compared to the active controls. In lay terms, concussed athletes started blinking sooner, had a greater discrepancy between timing of right and left eye lid movement, had a more open tonic lid position, and demonstrated hyperexcitability in their blink response.

Concussive events result in symptomatic deficits in attention, executive function, learning and memory. As such, tools have been developed to assess cognitive changes indicative of brain injury. However, concussive trauma is not restricted to the regions of the brain responsible for cognitive function, leading to the potential for missed or delayed diagnosis. Concussions result in diffuse axonal injury, which produces alterations in neurotransmitter levels, including dopamine.

Changes in dopamine levels and the time course over which those changes occur may explain the results found in this study in both the post-head impact and active control groups. Previous studies have shown that concussions elicit time dependent alterations in dopamine in various regions of the brain, with low levels found shortly after injury. It has also been shown that exercise alters neurotransmitter levels, including acute increases in dopamine and GABA. Not surprisingly, altered dopamine levels are found in several other neurological disorders, including Parkinson's disease, Huntington's disease, and schizophrenia. All of these disorders exhibit changes in individual latency and excitability of their blink reflexes. While the acute decrease in dopamine levels reported after a concussion and the acute increase in dopamine levels reported after exercise support the opposite trend in blink reflex measurements that the study observed between post-head impact and active control groups, neurotransmitter levels are unlikely to be the sole causative reason for the changes the study observed given the complex nature of a concussion. However, understanding the underlying mechanisms responsible for changes in the blink reflex is not necessary for the measurement to be a useful diagnostic tool.

Until now, field-side determination of whether an athlete has likely suffered a concussion has been based on the symptoms displayed. This study demonstrates the potential of the Blink Reflexometer to rapidly and objectively provide measurements of a primitive reflex that can assist the athletic trainer or medical personnel in determining the concussive status of an athlete. The ability to use reflex measurement to discriminate between individuals who have likely suffered a concussion and those who have simply been involved in active play will allow athletes to be removed from a game when appropriate. The fact that the reflex cannot be cheated should add a level of confidence.

The foregoing description provides illustration and description, but is not intended to be exhaustive or to limit the implementations to the precise form disclosed. Modifications and variations are possible in light of the above teachings or may be acquired from practice of the implementations.

While a series of blocks have been described with regard to FIG. 10, the order of the blocks may be modified in other implementations. Further, non-dependent blocks may be performed in parallel.

It will be apparent that devices and methods, as described above, may be implemented in many different forms of hardware, equipment, devices, systems, mechanical interconnections, and/or electrical interconnections in the implementations illustrated in the figures. The actual hardware, equipment, devices, systems, mechanical interconnections, and/or electrical interconnections used to implement these systems and methods is not limiting of the implementations—it being understood that hardware, equipment, devices, systems, mechanical interconnections, and/or electrical interconnections can be designed to implement the systems and methods based on the description herein. Further, certain portions, described above, may be implemented as a component that performs one or more functions.

Further, certain portions, described above, may be implemented as a component that performs one or more functions. A component, as used herein, may include hardware, such as a processor, an application specific integrated circuit (ASIC), or a field programmable gate array (FPGA), or a combination of hardware and software (e.g., a processor executing software).

In some aspects of the present invention, software executing the instructions provided herein may be stored on a non-transitory computer-readable medium, wherein the software performs some or all of the steps of one or more methods of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

It should be emphasized that the terms “comprises”/“comprising” when used in this specification are taken to specify the presence of stated features, integers, steps or components but does not preclude the presence or addition of one or more other features, integers, steps, components or groups thereof

Even though particular combinations of features are recited in the claims and/or disclosed in the specification, these combinations are not intended to limit the disclosure of the implementations. In fact, many of these features may be combined in ways not specifically recited in the claims and/or disclosed in the specification. Although each dependent claim listed below may directly depend on only one other claim, the disclosure of the implementations includes each dependent claim in combination with every other claim in the claim set.

No element, act, or instruction used in the present application should be construed as critical or essential to the implementations unless explicitly described as such. Also, as used herein, the article “a” and “an” are intended to include one or more items and may be used interchangeably with “one” or “more.” Where only one item is intended, the term “one” or similar language is used. Further, the phrase “based on” is intended to mean “based, at least in part, on” unless explicitly stated otherwise.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

1. A method for detecting changes associated with an eye generated in response to impaired neurological function, the method comprising:
when impaired neurological function is suspected in a subject, stimulating at least one facial region of a subject using at least one stimulator so as to cause an involuntarily blink response in the subject;
measuring at least one parameter of the response from one or both eyes resulting from the stimulating step; and
displaying information related to the at least one parameter.

2. The method of claim 1, wherein the at least one parameter comprises individual latency of one or both eyes of the subject.

3. The method of claim 1, wherein the at least one parameter comprises a differential latency between both eyes of the subject.

4. The method of claim 1, wherein the at least one parameter comprises counting oscillations of one or both eyes of the subject.

5. The method of claim 1, wherein the at least one parameter comprises measuring the tonic lid position of one or both eyes of the subject.

6. The method of claim 1, wherein the at least one parameter comprises changes in individual latency, differential latency, oscillations, and changes in tonic lid position.

7. The method of claim 1, further comprising the steps of:
comparing the at least one parameter to the at least one parameter measured at baseline; and
displaying information related to at least one difference between the at least one parameter and the at least one parameter measured at baseline.

8. The method of claim 1, wherein the suspected impaired neurological function is the result of a traumatic event, a head impact, or a mild traumatic brain injury.

9. The method of claim 8, further comprising determining if the subject has a mild traumatic brain injury.

10. The method of claim 1, wherein the at least one facial region comprises a region selected from the group consisting of the temple, the outer canthus, and the eye.

11-12. (canceled)

13. The method of claim 1, further comprising the step of:
comparing the at least one parameter to at least one parameter measured at baseline;
displaying information related to a difference between the at least one parameter and the at least one parameter measured at baseline;
determining based on the at least one parameter whether the subject has suffered a mild traumatic brain injury; and
indicating whether the subject has suffered a mild traumatic brain injury;
wherein the at least one parameter comprises measuring in one or both eyes of the subject changes in individual latency, differential latency, oscillations, and changes in tonic lid position.

14. An apparatus for detecting parameters associated with an eye upon delivering a stimulus thereto, the apparatus comprising:
at least one stimulator that provides a stimulus to one or both eyes of a subject;
a sensor configured to detect a parameter of one or both eyes; and
a user interface configured to control the at least one stimulator and display information detected by the sensor.

15. The apparatus of claim 14, wherein the at least one stimulator comprises a first unit and a second unit, wherein each unit is positioned to stimulate separate eyes of the subj ect.

16. The apparatus of claim 14, further comprising:
a detection device that is triggered when said at least one stimulator has provided a stimulus to an eye.

17. The apparatus of claim 14, wherein the at least one stimulator is aligned with at least one eye of a subject.

18. The apparatus of claim 14, wherein the sensor comprises a camera.

19. A system for detecting parameters associated with a blink response in a subject, comprising:
at least one stimulator that provides a stimulus to one or both eyes of a subject;
a sensor configured to detect a parameter of one or both eyes;
a user interface configured to control the at least one stimulator and display information detected by the sensor; and
a non-transitory computer-readable medium with instructions stored thereon, which when executed by a processor, perform steps comprising:
receiving a command from the user interface to begin measurement;
stimulating one or both eyes of a subject using at least one stimulator so as to cause an involuntarily response in the subject;
measuring at least one parameter of the response from one or both eyes resulting from the stimulating step; and
displaying on the user interface information related to the at least one parameter.

20. The system of claim 19, wherein the instructions further comprise the step of comparing the at least one parameter to at least one baseline parameter.

21. The system of claim 19, wherein the at least one parameter is selected from the group consisting of an individual latency, a differential latency, an oscillation count, and a tonic lid position.
